(19) United States
(12) Patent Application Publication
(10) Pub. No.: US 2012/0136046 A1
(43) Pub. Date: May 31, 2012

(54) METHOD OF TREATING AGE RELATED DISORDERS

(75) Inventors: Elizabeth Jane Mellor, Oxford (GB); Michael Youdell, Oxford (GB); Jonathan Ayling, Oxford (GB); Alexandre Akoulitchev, Ewelme (GB)

(73) Assignee: Chronos Therapeutics Limited, Oxford, Oxfordshire (GB)

(21) Appl. No.: 13/382,648

(22) PCT Filed: Jul. 8, 2010
(86) PCT No.: PCT/GB2010/051125

§ 371 Date: Feb. 17, 2012

(30) Foreign Application Priority Data
Jul. 8, 2009 (GB) 0911888.6

(51) Int. Cl. A61K 31/436; A61P 29/00; A61P 9/00; A61P 3/10; A61P 25/24; C12N 5/071; A61P 21/00; A61P 19/08; C12N 5/02; A61K 31/7088; C12N 1/18; C07D 498/18; A61P 25/28; A61P 3/04

(52) U.S. Cl. 514/44 A; 514/291; 435/375; 435/255.2; 435/420; 540/456; 435/366

(57) ABSTRACT

The present invention relates to the use of fujimycin for the treatment of a disorder related to the chronological and/or replicative life-span of a cell, and to methods for increasing the replicative life span of a cell, said method comprising disrupting the function of a polynucleotide or gene encoding a polypeptide comprising SEQ ID No: 1, 3 or 5.

### FIELD OF THE INVENTION

The current invention relates to methods for modulating the lifespan of a cell, and to methods and medicaments for the treatment of age related disorders.

### BACKGROUND

Yeast is a very good model system in which ageing and its regulation pathways can be studied with full relevance to humans¹.

There are two types of ageing. One is known as replicative ageing and is defined as the number of times a cell is capable of dividing. Each cell type has a characteristic replicative life span (RLS) which can be extended by interventions such as caloric restriction (CR), a well known method of extending life span in a wide range of organisms from yeast to mice. Replicative ageing is an important feature in the longevity of stem cells.

The second type of ageing is chronological ageing which is defined as the length of time a cell remains metabolically active and viable after it has stopped dividing. Many cells in mature mammals have stopped dividing and their CLS is an important feature of ageing. Like replicative ageing, chronological ageing is influenced by caloric restriction^{3, 4}. The sirtuins, for example, Sir2 in yeast, maintains the RLS of cells. There may be a minor influence on CLS which is specific to the genetic background of the strains. However, generally Sir2 does not direct regulation of CLS in wild type background^{5, 6.}

The links between ageing, age-related diseases and cellular lifespan regulation highlight the potential roles for regulators of cellular lifespan in the therapeutic treatment of many diseases including metabolic diseases, diabetes, inflammatory disorders, osteoporosis, cancer, cardiovascular diseases, etc.

The signals for nutrient availability, various growth factors, stress and energy status of the cell are integrated and translated into cellular growth by the complex TORC1 (target of rapamycin complex)⁹⁻¹¹, which includes the PI3-kinases Tor1 or Tor2¹². The signal from TORC1 is mediated by the substrates which include among others the kinases and transcription factors Sch9, Snf1 and Rtg2¹⁴. One of the targets of Snf1 kinase is Gcn5 (Kat2), a lysine acetyltransferase, and a component of both SAGA and SLIK complexes¹⁵⁻¹⁸. The SAGA and SLIK complexes regulate expression of groups of genes in response to cell growth signalling. Rtg2, a substrate of TORC1, is a known regulator of the mitochondrial retrograde response¹⁹, associated with ageing, and a subunit of the SLIK complex¹⁹. Another important subunit of both SAGA and SLIK complexes is Spt7, which is processed by Ubp8 in the context of the SLIK complex only^{17,20}. The activity of the SAGA complex is controlled separately by Spt8¹⁵.

Down regulation of TORC1 signalling by an alternative method of caloric restriction has been known to increase longevity in yeast and other organisms via major changes in gene expression^{9,10}. However, the mechanisms of such regulation have not been understood and are currently under investigation.

In yeast, mutation of the S6 kinase (Sch9) results in prolonged chronological and replicative lifespan. When the analogous gene is knocked out in mice or C. elegans an extension in lifespan is also observed.

In the current invention, the inventors have identified a further factor involved in the control of cellular lifespan and have shown that this has effects on both CLS and RLS.

The inventors have identified that Ydr026c in yeast is essential for sustaining high-order structure in chromatin. This high order structure has been implicated in playing an important role in ageing due to its role in the control of gene expression through epigenetics. This epigenetic control of genes is affected in ageing.

### SUMMARY

According to a first aspect of the present invention there is provided the use of fujimycin in the manufacture of a medicament for the treatment of an age related disorder.

Preferably, said age related disorder is selected from the group comprising a metabolic disorder; for example, disorders of carbohydrate metabolism (e.g. glycogen storage disease); disorders of amino acid metabolism (e.g. phenylketonuria, glutaric acidemia. etc.); disorders of organic acid metabolism (organic acidurias, e.g. alcaptonuria); disorders of fatty acid oxidation and mitochondrial metabolism (e.g. glutaric acidemia, type 2); disorders of purine and pyrimidine metabolism (e.g. Lesch-Nyhan syndrome); disorders of mitochondrial function (e.g. Kearns-Sayre syndrome); disorders of peroximal function (e.g. Zellweger syndrome) an inflammatory disorder, cardiovascular disease, diabetes type 1, diabetes type 2, artherosclerosis, Alzheimer's disease, dementia, conical depression, adipose disorders, including obesity, fat related metabolic disorders, muscular dystrophy, sarcopenia, cachexia and osteoporosis.

According to a second aspect of the present invention there is provided a method for increasing the replicative life span of a cell, said method comprising disrupting the function of a gene encoding a polypeptide comprising SEQ ID No: 1, 3 or 5, or a polypeptide having at least 70%, 80%, 90%, 95%, 97%, 98% or 99% identity thereto or a homologue thereof, or a polypeptide differing from SEQ ID NO: 1, 3 or 5 by one or several amino acid additions, deletions or substitutions.

According to a third aspect of the present invention there is provided a method of increasing the chronological lifespan of a cell, said method comprising disrupting the function of a gene encoding a polypeptide comprising SEQ ID No:1 or 3, or a polypeptide having at least 70%, 80%, 90%, 95%, 97%, 98% or 99% identity thereto or a homologue thereof, or a polypeptide differing from SEQ ID NO: 1, 3, or 5 by one or several amino acid additions, deletions or substitutions.

According to a fourth aspect of the present invention there is provided a method of increasing the chronological and/or replicative lifespan of a cell comprising treating said cell with fujimycin.

According to a fifth aspect of the present invention there is provided fujimycin for use in the treatment of an age related disorder

In one embodiment, preferably, said disorder is related to the chronological and/or replicative life-span of a cell,

In a further embodiment, said disorder is selected from the group comprising a metabolic disorder, for example, disorders of carbohydrate metabolism (e.g. glycogen storage disease); disorders of amino acid metabolism (e.g. phenylketonuria, glutaric acidemia. etc.); disorders of organic acid metabolism (organic acidurias, e.g. alcaptonuria); disorders of fatty acid oxidation and mitochondrial metabolism (e.g. glutaric acidemia, type 2); disorders of purine and pyrimidine metabolism (e.g. Lesch-Nyhan syndrome); disorders of mitochondrial function (e.g. Kearns-Sayre syndrome); disorders of peroximal function (e.g. Zellweger syndrome), an inflammatory disorder, cardiovascular disease, diabetes type 1, diabetes type 2, artherosclerosis, Alzheimer's disease, dementia, clinical depression, adipose disorders, including obesity, fat related metabolic disorders and muscular dystrophy, sarcopenia, cachexia and osteoporosis.

### DETAILED DESCRIPTION

The invention is based on the discovery by the inventors that yeast cells which do not express the polypeptide Ydr026c (SEQ ID NO: 3) have a greatly increased chronological lifespan when compared to cells which do express this polypeptide.

The invention also based on the further discovery that cells which do not express the polypeptide Ydr026c (SEQ ID NO: 3) also have an increased replicative lifespan when compared to cells expressing the polypeptide.

It will be apparent to the skilled person that the term chronological lifespan refers to the length of time a cells remains metabolically active and viable without division. It will be further apparent that the term replicative lifespan refers to the number of times a cell is capable of dividing. Therefore, it will be readily apparent that an increase relates to an increase in the length of time a cell remains viable without replicating or an increase in the number of times a cell can replicate before become senescent.

As used herein, the gene nomenclature YDR026C and OBD1 are synonymous and interchangeable. The gene encoding this polypeptide is located at the YDR026C locus in Saccharomyces cerevisiae.

As used herein, the term age related disorder relates to any disorder which has a factor of its etiology the age of the patient. It will be understood that age may only be one of a number of factors, which combined, result in the development of the disorder. Examples of such disorders include, but are not limited to metabolic disorders, for example, disorders of carbohydrate metabolism (e.g. glycogen storage disease); disorders of amino acid metabolism (e.g. phenylketonuria, glutaric acidemia. etc.); disorders of organic acid metabolism (organic acidurias, e.g. alcaptonuria); disorders of fatty acid oxidation and mitochondrial metabolism (e.g. glutaric acidemia, type 2); disorders of purine and pyrimidine metabolism (e.g. Lesch-Nyhan syndrome); disorders of mitochondrial function (e.g. Kearns-Sayre syndrome); disorders of peroximal function (e.g. Zellweger syndrome), type 2 diabetes, endocrine disorders, for example thyroid disorders, cancer, inflammatory disorders, autoimmune disorders, cardiovascular disease, diabetes type 1, artherosclerosis, including Alzheimer's disease, dementia, clinical depression, adipose disorders, including obesity, fat related metabolic disorders and muscular dystrophy, sarcopenia, cachexia and osteoporosis.

According to a different aspect of the present invention there is provided the use of fujimycin in the manufacture of a medicament for the treatment of an age related disorder.

Preferably said disorder is selected from the group comprising a metabolic disorder for example, disorders of carbohydrate metabolism (e.g. glycogen storage disease); disorders of amino acid metabolism (e.g. phenylketonuria, glutaric acidemia. etc.); disorders of organic acid metabolism (organic acidurias, e.g. alcaptonuria); disorders of fatty acid oxidation and mitochondrial metabolism (e.g. glutaric acidemia, type 2); disorders of purine and pyrimidine metabolism (e.g. Lesch-Nyhan syndrome); disorders of mitochondrial function (e.g. Kearns-Sayre syndrome); disorders of peroximal function (e.g. Zellweger syndrome), an inflammatory disorder, cardiovascular disease, diabetes type 1, diabetes type 2, artherosclerosis, Alzheimer's disease, dementia, clinical depression, adipose disorders, including obesity, fat related metabolic disorders, muscular dystrophy, sarcopenia, cachexia and osteoporosis.

In one preferred embodiment, the disorder is selected from Alzheimer's disease, dementia, clinical depression.

In another preferred embodiment, the disorder is selected sarcopenia or cachexia.

Addition of fujimycin to cells results in an increased CLS and increased RLS. It will be apparent to the skilled person that such an increase in the CLS and/or RLS of cells may have implications for the treatment of disorders associated with ageing.

The medicaments comprise a therapeutically effective amount of the agent(s) of the present invention and preferably a pharmaceutically acceptable carrier, diluent or excipient (including combinations thereof).

The medicaments may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The medicaments may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the medicament. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the medicament of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the medicaments can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

If the agent is a protein, then said protein may be prepared in situ in the subject being treated. In this respect, nucleotide sequences encoding said protein may be delivered by use of nonviral techniques (e.g. by use of liposomes) and/or viral techniques (e.g. by use of retroviral vectors) such that the said protein is expressed from said nucleotide sequence.

A “stable” formulation is one in which the polypeptide or protein therein essentially retains its physical and chemical stability and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 2990 (1993). Stability can be measured at a selected temperature for a selected time period. For rapid screening, the formulation of interest may be kept at 40° C. for 1 week to 1 month, at which time stability is measured. The extent of aggregation following lyophilization and storage can be used as an indicator of peptide and/or protein stability. For example, a “stable” formulation is one wherein less than about 10% and preferably less than about 5% of the polypeptide or protein is present as an aggregate in the formulation. An increase in aggregate formation following lyophilization and storage of the lyophilized formulation can be determined. For example, a “stable” lyophilized formulation may be one wherein the increase in aggregate in the lyophilized formulation is less than about 5% or less than about 3%, when the lyophilized formulation is incubated at 40° C. for at least one week. Stability of the fusion protein formulation may be measured using a biological activity assay such as a binding assay as described herein.

According to a further aspect, there is provided a method for increasing the replicative life span of a cell, said method comprising disrupting the function of a polynucleotide or gene encoding a polypeptide comprising SEQ ID No: 1, 3 or 5, or a polypeptide having at least 70%, 80%, 90%, 95%, 97%, 98% or 99% identity thereto or a homologue thereof or a polypeptide differing from SEQ ID NO: 1, 3 or 5 by one or several amino acid additions, deletions or substitutions.

In preferred embodiments, the polynucleotide or gene comprises the nucleotide sequence shown in SEQ ID NO: 4, 6 or 7; or a sequence which has at least 70%, 80%, 90%, 95%, 97%, 98% or 99% identity thereto; or differs from SEQ ID NO: 4, 6 or 7 due to degeneracy of the genetic code; or differs from SEQ ID NO: 4, 6 or 7 by one or several nucleic acid additions, deletions or substitutions.

In a preferred embodiment, the cell is a stem cell. More preferably, the cell is a mammalian stem cell. Most preferably, the cell is a human stem cell.

In a further preferred embodiment, the cell is an induced pluripotent stem cell.

It will be understood that the term cell as used in relation to the present invention, refers can refer to both plant and animal cells and to both a cell or population of cells maintained ex vivo, or alternatively, and where appropriate, to an in vivo cell or population of cells.

It should be noted that SEQ ID NOs:1 and 5 (TTF1) and SEQ ID NO:3 (Ydr026c) are human and yeast homologues of the same protein. Furthermore, SEQ ID NO: 3 (this work) and the mouse homolog (Shiue, C-N, et al, (2009) Oncogene, 28, 1833-1842) have both been shown to regulate gene loop formation.

It will be further understood that SEQ ID NOs:1 and 5 are splice variants of human TTF1.

According to a further aspect of the present invention, there is provided a method of increasing the chronological lifespan of a cell, said method comprising disrupting the function of a polynucleotide or gene encoding a polypeptide comprising SEQ ID No:1, 3 or 5; or a polypeptide having at least 70%, 80%, 90%, 95%, 97%, 98% or 99% identity thereto or a homologue thereof; or a polypeptide differing from SEQ ID NO: 1, 3 or 5 by one or several amino acid additions, deletions or substitutions.

In preferred embodiments, the polynucleotide or gene comprises the nucleotide sequence shown in SEQ ID NO: 4, 6 or 7; or a sequence which has at least 70%, 80%, 90%, 95%, 97%, 98% or 99% identity thereto; or differs from SEQ ID NO: 4, 6 or 7 due to degeneracy of the genetic code; or differs from SEQ ID NO: 4, 6 or 7 by one or several nucleic acid additions, deletions or substitutions.

It will be understood that the homologue can be an autologous or heterologous peptide i.e from the same or a different species.

In the present context, a homologue is taken to include an amino acid sequence which may be at least 50, 60, 70, 75, 80, 85 or 90% identical, preferably at least 95, 97%, 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed.

Calculation of maximum % homology therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

It will be understood that the term disrupting the function refers to disrupting the expression of the polynucleotide or gene or disrupting the activity of the encoded polypeptide. It will be further understood that any stage of gene expression between initiation of transcription and production of a mature protein can be disrupted. The skilled person will understand that this will include epigenetic means of controlling gene expression through controlling chromatin structure as well as transcriptional, translational and post translation means of controlling gene expression.

It will be understood that by disrupting expression of a gene as used herein is meant preventing or inhibiting production of a functional polypeptide by any means known in the art and that disrupting the activity of the encoded polypeptide refers to disrupting interaction of the functional polypeptide with one or more of it's binding partners such that the polypeptide does not perform it's function. The production or function may be fully or partially prevented. In one embodiment, preferably the production or function of the gene product is fully prevented, i.e. there is no active gene product. In some instances the production or function of the gene product may be disrupted such that there is only about 5%, about 10% about 20%, about 30%, about 50%, about 60%, about 70%, about 80%, about 90% or about 95% of the wild type level of expression remaining.

As used herein by inhibiting production of a functional polypeptide it is meant that the production of the gene product may be prevented or inhibited by (a) knocking out said gene; (b) post-transcriptionally silencing said gene through for example the use of iRNA or antisense RNA (gene silencing); (c) transcriptionally silencing said gene by, for example, epigenetic techniques; (d) preventing or altering the function of the gene product by the introduction of at least one point mutation; (e) post translationally inactivating the gene product.

In one preferred embodiment, expression of the gene or homologue is disrupted by iRNA.

Preferably, the cell is transformed with a plasmid/vector encoding an iRNA under control of a promoter. It will be apparent that this promoter may be a constitutive promoter and/or a tissue specific promoter.

As used herein the term iRNA refers to RNA interference (RNAi). This is a method of post-transcriptional gene silencing (PTGS) in eukaryotes induced by the direct introduction of dsRNA (Fire A, et al., (1998)).

In a further preferred embodiment expression of the gene is disrupted at the transcriptional/DNA level. Preferably, said disruption is effected by insertion of at least one nucleotide into the gene or deletion of at least one nucleotide from the gene.

In a further embodiment, the disruption of the gene is effected by introduction of at least one point mutation.

It will be understood that in the case of disruption of the interaction of the polypeptide with one or more of it's binding partners. this disruption can be by any suitable means, for example, competitive inhibition, non-competitive inhibition, mixed inhibition or uncompetitive inhibition.

According to the present invention there is provided a method of increasing the chronological and/or replicative lifespan of a cell comprising treating said cell with fujimycin.

As discussed above, the cell may be a plant or animal cell. Preferably, said cell is an animal cell. In one preferred embodiment, the cell is a mammalian cell. More preferably, said cell is a human cell

Further provided by the present invention is fujimycin for use in the treatment of an age related disorder.

Preferably, the disorder is related to the chronological and/or replicative lifespan of a cell,

Preferably, said disorder selected from the group comprising a metabolic disorder, for example, disorders of carbohydrate metabolism (e.g. glycogen storage disease); disorders of amino acid metabolism (e.g. phenylketonuria, glutaric acidemia. etc.); disorders of organic acid metabolism (organic acidurias, e.g. alcaptonuria); disorders of fatty acid oxidation and mitochondrial metabolism (e.g. glutaric acidemia, type 2); disorders of purine and pyrimidine metabolism (e.g. Lesch-Nyhan syndrome); disorders of mitochondrial function (e.g. Kearns-Sayre syndrome); disorders of peroximal function (e.g. Zellweger syndrome), an inflammatory disorder, cardiovascular disease, diabetes type 1, diabetes type 2, artherosclerosis, Alzheimer's disease, dementia, clinical depression, adipose disorders, including obesity, fat related metabolic disorders muscular dystrophy, sarcopenia, cachexia and osteoporosis.

In one preferred embodiment, the disorder is selected from Alzheimer's disease, dementia, clinical depression.

In another preferred embodiment, the disorder is selected sarcopenia or cachexia.

The skilled person will be aware that Fujimycin (FK-506) is an immunosuppressive antibiotic drug which is mainly used after organ transplantation. It is believed that Fujimycin acts by inhibiting the activity of calcineurin (Griffith, J. P., et al, [1995] Cell, 82(3), 507-522).

Alzheimer's disease is not only a disease of ageing but also one which should be reversible by disruption of pathways which regulate ageing. For example, mutation of Daf2 in C. elegans can slow the progression of C. elegans models of Alzheimer's. This mutation disrupts IGF-1 signalling and results in an extension of lifespan. Analogous experiments carried out in mice suggest the same hypothesis. Alzheimer's disease is also linked to the accumulation of toxic protein aggregates. Compounds, such as FK-506 (fujimycin) which extend chronological lifespan can ameliorate the accumulation of these aggregates, and so may be useful in the treatment of Alzheimer's and related indications such as dementia and clinical depression.

Unless defined otherwise, all technical and scientific terms used herein have the same meaning as commonly understood by one of ordinary skill in the art. Practitioners are particularly directed to Current Protocols in Molecular Biology (Ausubel) for definitions and terms of the art. Abbreviations for amino acid residues are the standard 3-letter and/or 1-letter codes used in the art to refer to one of the 20 common L-amino acids.

It is further noted that, as used in this specification, the singular forms “a,” “an,” and “the” include plural referents unless expressly and unequivocally limited to one referent. The ter “or” is used interchangeably with the term “and/or” unless the context clearly indicates otherwise.

Also, the terms “portion” and “fragment” are used interchangeably to refer to parts of a polypeptide, nucleic acid, or other molecular construct.

“Polypeptide” and “protein” are used interchangeably herein to describe protein molecules that may comprise either partial or full-length proteins.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues.

The term “plasmid/vector” in one embodiment refers to a nucleic acid molecule that may be used to transport a second nucleic acid molecule into a cell. In one embodiment, the vector allows for replication of DNA sequences inserted into the vector. The vector may comprise a promoter to enhance expression of the nucleic acid molecule in at least some host cells. Vectors may replicate autonomously (extrachromasomal) or may be integrated into a host cell chromosome. In one embodiment, the vector may comprise an expression vector capable of producing a protein derived from at least part of a nucleic acid sequence inserted into the vector. As used herein, an “effective amount” means the amount of an agent that is effective for producing a desired effect in a subject. The term “therapeutically effective amount” denotes that amount of a drug or pharmaceutical agent that will elicit therapeutic response of an animal or human that is being sought. The actual dose which comprises the effective amount may depend upon the route of administration, the size and health of the subject, the disorder being treated, and the like.

The term “pharmaceutically acceptable carrier” as used herein may refer to compounds and compositions that are suitable for use in human or animal subjects, as for example, for therapeutic compositions administered for the treatment of a disorder or disease of interest.

The invention will now be described in further detail with reference to the figures in which:—

### FIGURES

FIG. 1 shows that the Ydr026c gene product produced from the YDR026C locus in yeast is directly implicated in formation of high-order structure on a gene as part of epigenetic regulation in yeast.

FIG. 2A shows the extension of chronological life span under anaerobic conditions in ydr026c depleted mutant, but not in the wild type of fob1 depleted mutant. FIG. 2B shows the same results as calibrated growth curves.

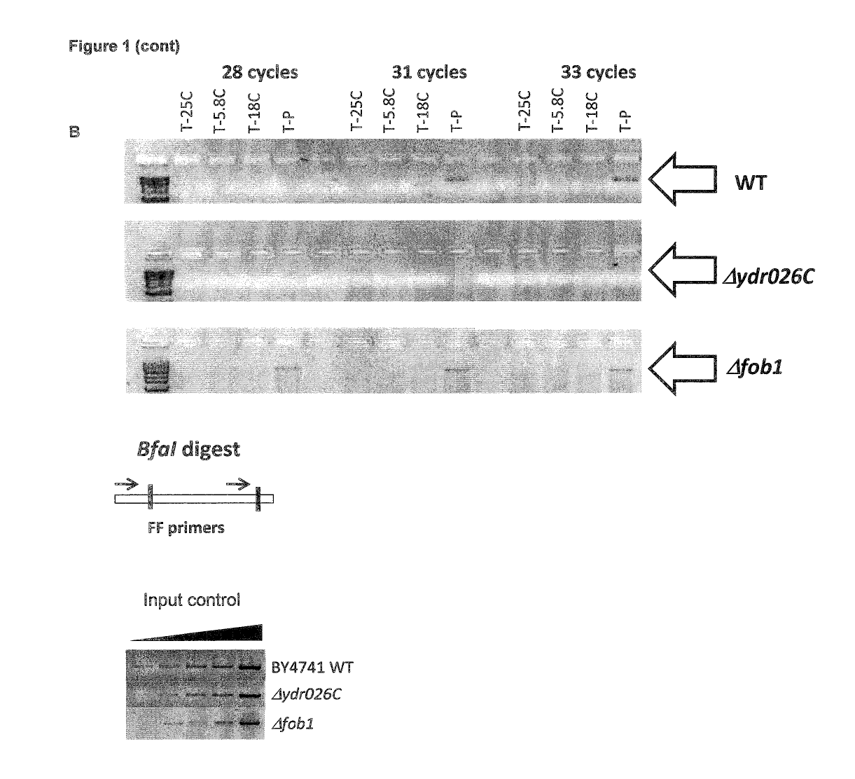

FIG. 3 shows the chronological life span for wild type (BY4741), ydr026cΔ and fob1Δ yeast strains under aerated (top panel) and deoxygenated (bottom panel) conditions.

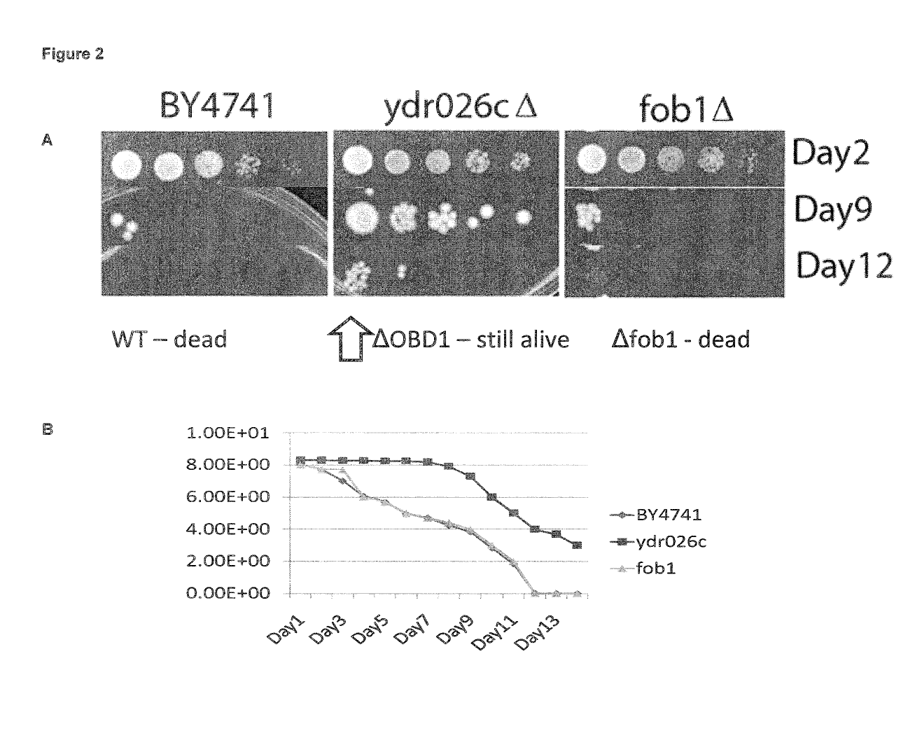

FIG. 4 shows the effect of fujimycin on chronological life span.

FIG. 5 shows the effect of FK-506 on the lifespan of C. elegans

FIG. 6 shows the effect of FK-506 on the rate of cell division in the human fibroblast cell line MRC5.

FIG. 7 shows the effects of deleting obd1 and sir2 on the chronological lifespan of cells.

### EXAMPLES

### Example 1

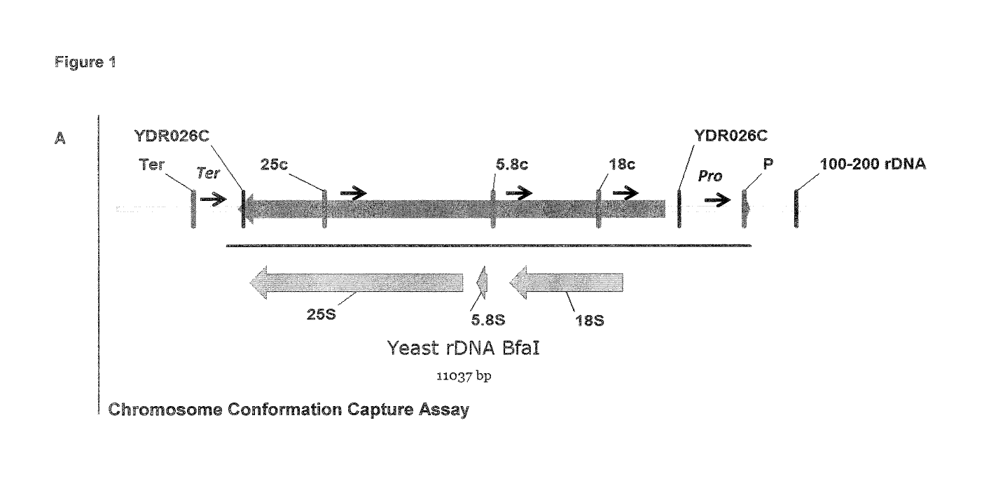

FIG. 1 shows the effect of the YDR026C gene product on the high-order structure of a gene this forms part of the epigenetic regulation of stability of the locus, as exemplified by rDNA. Loss of Ydr026c and its function leads to the loss of epigenetic high order chromosomal structures, as measured by chromosome conformation capture assay (FIG. 1B). Loss of normal high order structure observed in wt is associated with epigenetic deregulation and loss of locus stability. Loss of Fob1, the interaction partner of Ydr026c, leads to the increased stability of the high order structure.

FIG. 1A shows the yeast rDNA locus with two binding sites for YDR026C upstream and downstream of the locus, the position of Bfal restriction sites used in standard Chromosome Conformation Capture Assay (vertical lines), and the position of PCR primers labelled Ter, 25c, 5.8c, 18c and Pro (small arrows), also shown are the rRNA transcripts made from the locus (block arrows).

FIG. 1B shows the results of Chromosome Conformation Capture Assay (3C) assay. In WT this shows the production of a specific product band after 33 cycles with primer pair Ter-Pro in wild type. The same product is seen after 28 cycles in a fob1 deleted strain. It should be noted that no product is seen even after 33 cycles in ydr026C deleted strain.

This indicates that loss of Ydr026c leads to the loss of the characteristic epigenetic high-order structure observed in wild type normal transcription from the rDNA locus in yeast. It also indicates that loss of fob1, which interacts with Ydr026c leads to high-stability of the high-order structures, making them easy to detect at early PCR cycles of the 3C assay.

### Example 2

Cells were grown for the indicated number of days without aeration (oxygen deprivation) in 2% glucose containing medium and plated in 10-fold dilutions to monitor viability.

This experiment demonstrates that the Ydr026c depleted mutant has extended life span, as compared to the wild type or fob1 depleted mutant. The figure shows that after 12 days in anaerobic culture, the wild type and Δfob1 strains are no longer viable whereas the ydr026cΔ strain still shows growth.

### Example 3

In this experiment the wild type, ydr026cΔ and fob1Δ yeast cells were plated on to growth media after the indicated number of days culture in 3% glucose media under either aerated or deoxygenated (AN) culture conditions. As can be seen from the figure, under both aerobic and anaerobic conditions, the ydr026cΔ mutant shows increased chronological life span of 33 days and 20 days respectively.

### Example 4

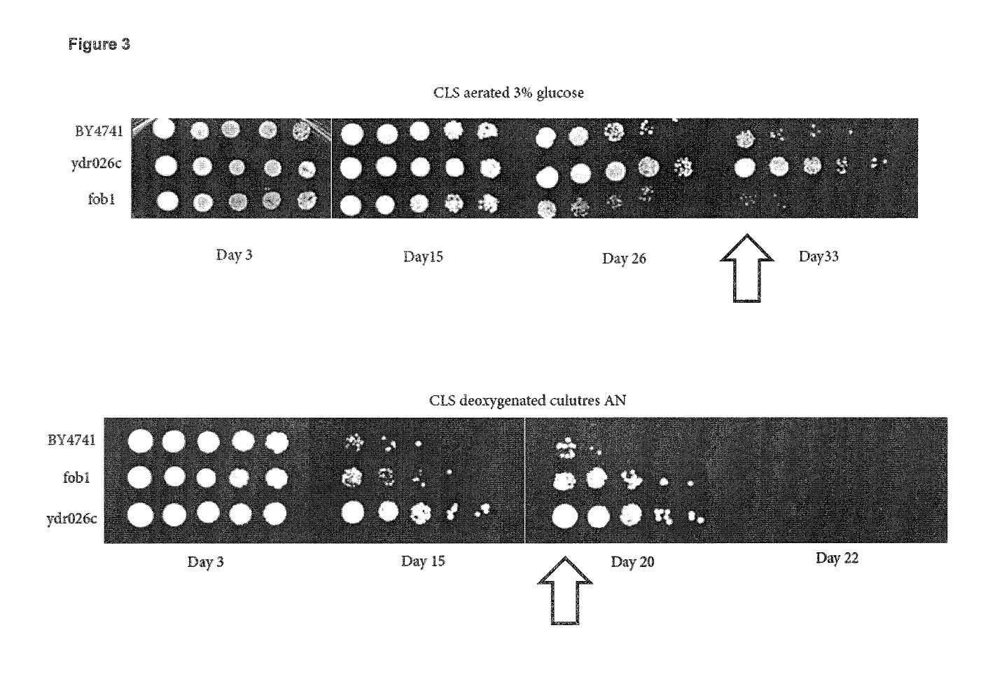

As can be seen from FIG. 4, FK-506 (fujimycin) prolongs the chronological lifespan of S. cerevisiae. Addition of Fujimycin to the culture medium at 0.1 ng/ml has no significant effect on the chronological lifespan of the cells compared to the control, whereas the addition of 1 ng/ml or more has a significant effect increasing the median lifespan of the yeast cells compared to the control. Lifespan was measured using the outgrowth method described by Murakami, C. and Kaeberlein, M., (2009) J. Vis. Exp, 27.

Briefly, chronological lifespan of yeast refers to the profile of viability of an ageing yeast culture over time. To measure this a yeast culture is grown in liquid media until the glucose carbon source is exhausted and the cells stop dividing. At this point the proportion of cells which are alive and able to divide is measured by observing the outgrowth characteristics of a fresh inoculate of the ageing culture using a Bioscreen™ C machine (Oy Growth Curves Ab Ltd, Finland). Viabilities at various time points are compared to determine the chronological lifespan of the culture.

### Example 5

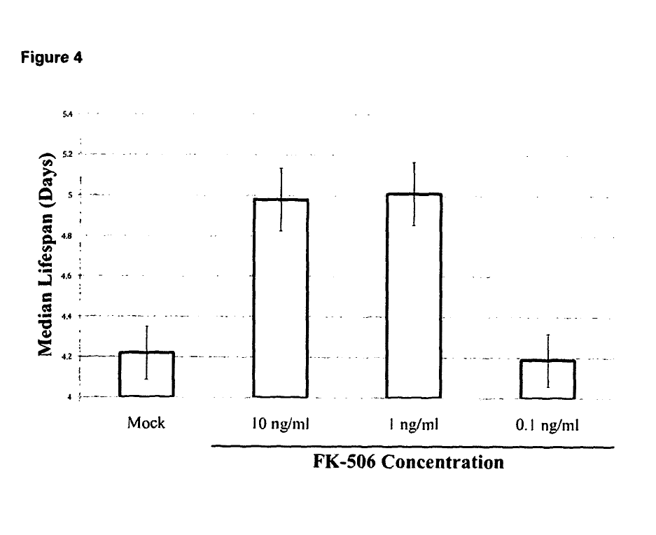

As can be seen from FIG. 5, addition of FK-506 prolongs the lifespan of C. elegans in a dose dependent manner with the addition of 4μ/ml resulting in a significant increase in the viability of the organisms (chronological lifespan). FK-506 was added to C. elegans during the timed egg laying stage and lifespan was measured as described in Sutphin, G. L., and Kaeberlein, M., (2009) J. Vis. Exp, 27. Briefly this method comprises synchronising a population of worms and measuring the percentage of living worms at a range of time points, scoring live worms by their ability to respond to physical stimulus.

### Example 6

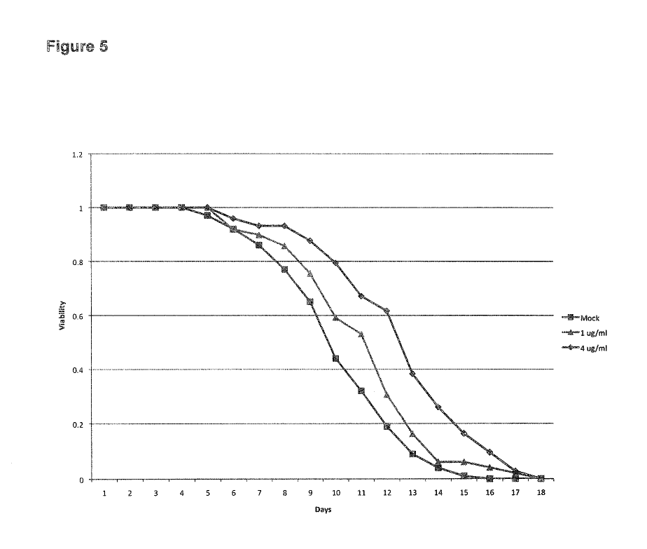

FIG. 6 shows that FK-506 appears to reverse age dependent reduction in cell division rate in the MRC5 human fibroblast cell line. This indicates that FK506 increases replicative lifespan of these cells. The cell division rate was measured using the method of Fairweather, D. S., M. Fox, and G. P. Margison, (1987), Exp Cell Res, 168(1): p. 153-9. Briefly, the replicative lifespan of MRC5 cells are measured by growing them on the surface of dishes until the reach confluencey whereupon they are split at a 1:2 ratio (i.e. diluted 1 in 2). The number of times the culture is split is equal to that of the number of divisions the cells are able to undertake which is the cultures replicative lifespan. MRC5 cells are only able to divide a finite number of times, and as they reach the end of their life the time they take to undergo cell division increases. In this instance the rate of cell division is used as a measure of age.

As can be seen, the addition of FK-506 after 39 divisions results in a reduction of the division time compared to a control having no FK-506 added.

At higher concentrations, the FK506 becomes toxic to the cells and thus limits the cells replicative capacity.

The data suggests that FK-506 is able to delay the onset of ageing in both humans and C. elegans. Ageing in C. elegans is primarily the ageing of the muscle and intestinal cells. The data shows that FK-506 is extending the lifespan of C. elegans and must therefore delay ageing in these tissues. This indicates that FK-506 will delay the effects of ageing of human muscle cells and will be useful in the treatment of disorders such as sarcopenia. A development of this disease is muscle loss associated with bed rest or chemotherapy (cachexia) which is mechanistically believed to be a very similar process.

The data further suggest that FK-506 is able to delay the onset of senescence and so may be useful in increasing the efficiency of induced pluripotent stem cells and directly converted cells, which may be used in stem cell therapies.

### Example 7

Increase in Replicative life span (RLS) of dividing stem cells by deletion of the YDR 026c locus. The Replicative lifespan was measured using a micromanipulator (Singer Instruments). This involves physically dissecting each daughter cell produced during the lifetime of a single mother cell growing on a YPD agar medium (2% glucose, 2% yeast extract, 1% bactopeptone, 2% agar). The data from 10-50 mother cells was averaged to produce a mean life-span for each strain and its wild-type parent. Care was taken to ensure that the starting virgin cells were produced from small (therefore young) mother cells. [Kaeberlein, M., Kirkland, K. T., Fields, S. & Kennedy, B. K., Genes determining yeast replicative life span in a long-lived genetic background. Mech Ageing Dev 126, 491-504 (2005) and Kaeberlein, M. et al., Regulation of yeast replicative life span by TOR and Sch9 in response to nutrients. Science 310, 1193-1196 (2005)].

TABLE 1

| Yeast Strain | Obd1 Expression | RLS |
|---|---|---|
| BY4741 | Yes | 25 |
| ydr026cΔ | No | 30 |

As can be seen from table 1, for BY4741 (wild type control) the mean lifespan is 25 generations and for the strain lacking Ydr026c it is 30 generations.

### Example 8

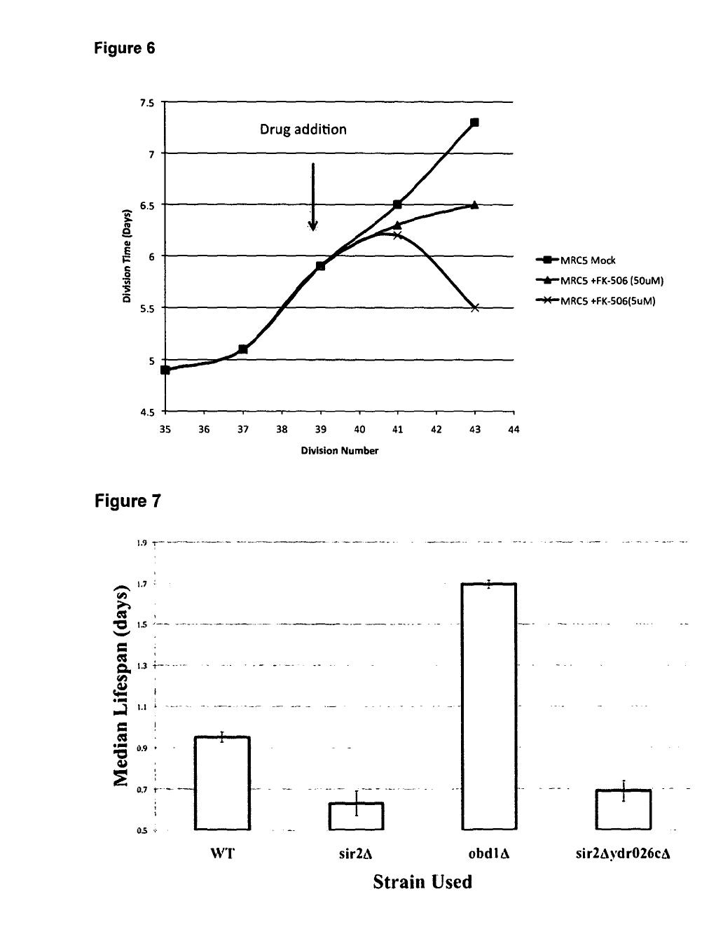

Shown below is a model based on the data shown in FIG. 7. The model shows that Obd1 deletion dependent lifespan extension is due to the inhibitory effect of OBD1 on Sir2. This suggests that any mechanism of extending lifespan life by activating Sir2 will be limited by inhibition by Obd1.

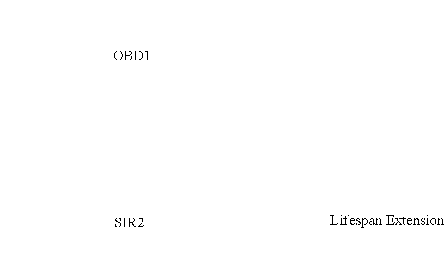

FIG. 7 shows the median lifespan of the yeast strain BY474 1 in which either obd1, sir2 or both obd1 and sir2 have been disrupted. Lifespan was calculated using the previously described Bioscreen™ C based protocol. Yeast strains were produced as described by Longtine et al (Yeast vol. 14 p. 953 Additional modules for versatile and economical PCR based gene deletion and modification in Saccharomyces cerevisiae).

The data clearly shows that disruption of obd1 significantly increases chronological lifespan whereas disruption of sir2 significantly decreases chronological lifespan when compared to wildtype. Interestingly, a double mutant in which both obd1 and sir2 are disrupted results in a significant decrease in chronological lifespan.

Furthermore, the data shown in Table 2 indicate the epistatic effect of obd1 on sir2 activity

| Strain | CLS | ERCs | Recombination |
|---|---|---|---|
| WT | Normal | Normal | Normal |
| sir2Δ | Low | High | High |
| obd1 Δ | High | Low | Low |
| obd1Δsir2Δ | Low | Very High | Very High |

Recombination rate was measured by measuring the number of ADE+ revertants by observing colour changes in yeast colonies. ERC's are extra-ribosomal-chromosomes which occur due to aberrant recombination resulting is short circular DNA sequences being present in the nuclei, levels of which can be detected by Southern Blot. This data shows that in the absence of obd1, sir2 expression results in an increased chronological lifespan and a decrease in the rate of recombination.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

1. Sinclair, D. A., Lin, S. J. & Guarente, L. Life-span extension in yeast. Science 312, 195-197; author reply 195-197 (2006).
2. Sinclair, D. A. & Guarente, L. Extrachromosomal rDNA circles—a cause of aging in yeast. Cell 91, 1033-1042 (1997).
3. Cohen, H. Y. et al. Calorie restriction promotes mammalian cell survival by inducing the SIRT1 deacetylase. Science 305, 390-392 (2004).
4. Anderson, R. M. et al. Yeast life-span extension by calorie restriction is independent of NAD fluctuation. Science 302, 2124-2126 (2003).
5. Kennedy, B. K., Smith, E. D. & Kaeberlein, M. The enigmatic role of Sir2 in aging. Cell 123, 548-550 (2005).
6. Smith, D. L., Jr., McClure, J. M., Matecic, M. & Smith, J. S. Calorie restriction extends the chronological lifespan of Saccharomyces cerevisiae independently of the Sirtuins. Aging cell 6, 649-662 (2007).
7. Kaeberlein, M., Kirkland, K. T., Fields, S. & Kennedy, B. K. Genes determining yeast replicative life span in a long-lived genetic background. Mech Ageing Dev 126, 491-504 (2005).
8. Kaeberlein, M. & Powers, R. W., 3rd Sir2 and calorie restriction in yeast: a skeptical perspective. Ageing Res Rev 6, 128-140 (2007).
9. J. R. Rohde, R. Bastidas, R. Puria et al., Nutritional control via Tor signaling in Saccharomyces cerevisiae. Curr Opin Microbiol 11, 153-160 (2008).
10. R. W. Powers, 3rd, M. Kaeberlein, S. D. Caldwell et al., Extension of chronological life span in yeast by decreased TOR pathway signaling. Genes Dev 20, 174-184 (2006).
11. N. D. Bonawitz, M. Chatenay-Lapointe, Y. Pan et al., Reduced TOR signaling extends chronological life span via increased respiration and upregulation of mitochondrial gene expression. Cell metabolism 5, 265-277 (2007).
12. M. C. Lorenz and J. Heitman, TOR mutations confer rapamycin resistance by preventing interaction with FKBP12-rapamycin. J Biol Chem 270, 27531-27537 (1995).
13. Karl W. Henry, Anastasia Wyce, Wan-Sheng Lo et al., Transcriptional activation via sequential histone H2B ubiquitylation and deubiquitylation, mediated by SAGA-associated Ubp8. Genes Dev. 17, 2648-2663 (2003).
14. C. De Virgilio and R. Loewith, Cell growth control: little eukaryotes make big contributions. Oncogene 25, 6392-6415 (2006).
15. a E. Sterner, R. Belotserkovskaya, and S. L. Berger, SALSA, a variant of yeast SAGA, contains truncated Spt7, which correlates with activated transcription. Proc Natl Acad Sci U S A 99, 11622-11627 (2002).
16. D. E. Sterner, P. A. Grant, S. M. Roberts et al., Functional organization of the yeast SAGA complex: distinct components involved in structural integrity, nucleosome acetylation, and TATA-binding protein interaction. Mol Cell Biol 19, 86-98 (1999).
17. P. Y. Wu and F. Winston, Analysis of Spt7 function in the Saccharomyces cerevisiae SAGA coactivator complex. Mol Cell Biol 22, 5367-5379 (2002).
18. P. A. Grant, L. Duggan, J. Cote et al., Yeast Gcn5 functions in two multisubunit complexes to acetylate nucleosomal histones: characterization of an Ada complex and the SAGA (Spt/Ada) complex. Genes Dev 11, 1640-1650 (1997).
19. Z. Liu and R. A. Butow, Mitochondrial retrograde signaling. Annu Rev Genet 40, 159-185 (2006).
20. S. M. Hoke, G. Liang, A. I. Mutiu et al., C-terminal processing of yeast Spt7 occurs in the absence of functional SAGA complex. BMC Biochem 8, 16 (2007).
21. H. Santos-Rosa, R. Schneider, A. J. Bannister et al., Active genes are tri-methylated at K4 of histone H3. Nature 419, 407-411 (2002).

```
Human TTF 1
SEQ ID NO: 1
MEGESSRFEI HTPVSDKKKK KCSIHKERPQ KHSHEIFRDS SLVNEQSQIT
RRKKRKKDFQ HLISSPLKKS RICDETANAT STLKKRKKRR YSALEVDEEA
GVTVVLVDKE NINNTPKHFR KDVDVVCVDM SIEQKLPRKP KTDKFQVLAK
SHAHKSEALH SKVREKKNKK HQRKAASWES QRARDTLPQS ESHQEESWLS
VGPGGEITEL PASAHKNKSK KKKKKSSNRE YETLAMPEGS QAGREAGTDM
QESQPTVGLD DETPQLLGPT HKKKSKKKKK KKSNHQEFEA LAMPEGSQVG
SEVGADMQES RPAVGLHGET AGIPAPAYKN KSKKKKKKSN HQEFEAVAMP
ESLESAYPEG SQVGSEVGTV EGSTALKGFK ESNSTKKKSK KRKLTSVKRA
RVSGDDFSVP SKNSESTLFD SVEGDGAMME EGVKSRPRQK KTQACLASKH
VQEAPRLEPA NEEHNVETAE DSEIRYLSAD SGDADDSDAD LGSAVKQLQE
FIPNIKDRAT STIKRMYRDD LERFKEFKAQ GVAIKFGKFS VKENKQLEKN
VEDFLALTGI ESADKLLYTD RYPEEKSVIT NLKRRYSFRL HIGRNIARPW
KLIYYRAKKM FDVNNYKGRY SEGDTEKLKM YHSLLGNDWK TIGEMVARSS
LSVALKFSQI SSQRNRGAWS KSETRKLIKA VEEVILKKMS PQELKEVDSK
LQENPESCLS IVREKLYKGI SWVEVEAKVQ TRNWMQCKSK WTEILTKRMT
NGRRIYYGMN ALRAKVSLIE RLYEINVEDT NEIDWEDLAS AIGDVPPSYV
QTKFSRLKAV YVPFWQKKTF PEIIDYLYET LPLLKEKLET KMMEKKGTKI
QTPAAPKQVF PFRDIFYYED DSEGEDIEKE SEGQAPCMAH ACNSSTLGGQ
GRWII
Human TTF1 DNA
SEQ ID NO: 2
agcagcactt ccgggttggg agaaaggtgg cggcgctttc ggagggaata
aaatggaagg agaatcaagc agatttgaaa tccacactcc agtttctgac
aagaaaaaga aaaagtgttc tatacataag gaaagacctc agaaacattc
ccacgaaatt ttcagagact cctccctggt gaatgaacag tctcaaataa
ctaggaggaa aaagaggaaa aaagatttcc agcatctcat ttcttctcct
ttgaaaaaat ccagaatctg tgatgagact gcaaatgcca cttccacact
caaaaagaga aaaaagagaa gatatagtgc tttggaggtg gacgaggaag
caggtgttac agttgtcctt gtggataaag aaaatattaa caacacacca
aagcatttta gaaaggatgt tgatgttgtt tgtgttgata tgagcataga
acagaagtta ccaagaaagc ctaaaacaga caaatttcag gtacttgcta
agtcacatgc acataaatca gaagccctgc acagtaaagt tagggagaaa
aagaataaaa agcatcagag gaaagctgca tcctgggaga gccagcgggc
aagggacacc ctgcctcagt cagaatcccc caggaggaga tcctggcttt
ctgtgggtcc agggggtgaa attacagaac taccagcatc tgctcataaa
aacaagtcta agaaaaaaaa gaaaaagtcc agtaaccggg aatatgagac
actggccatg cctgaaggat cgcaagcagg cagagaggcc gggactgata
tgcaggaatc ccagcctact gtgggcttgg atgatgaaac tccacaacta
ctaggaccta ctcacaaaaa aaagtctasag aaaaaaaga agaaaaagtc
caatcaccag gaatttgagg cattggccat gcctgaagga tcacaagtgg
gcagtgaggt tggggctgat atgcaggaat cccggcctgc tgtgggcctg
catggtgaaa ctgcaggaat accagcacct gcttataaaa acaagtctaa
gaaaaaaaag aaaaagtcca atcaccagga atttgaggca gtggccatgc
ctgagagcct cgagagtgca taccctgaag gatcacaggt gggcagtgag
gttgggactg tggaaggcag tacagctctt aaagggttca aggaatccaa
cagtacaaag aagaagtcta agaaaaggaa gcttacgtct gtcaaaaggg
cacgagtgtc tggtgatgat ttttcagtgc ccagtaagaa ctctgagagc
acactctttg attcagtaga aggtgatggc gccatgatgg aagaaggtgt
gaaatctagg ccccgacaaa agaaaaccca ggcctgtttg gcaagcaagc
acgtgcaaga ggcgccaagg ttagaacctg caaatgaaga acacaatgtg
gaaacagctg aagattccga aataagatac ttatctgcag attcaggaga
tgccgatgat tcagatgcgg atttgggttc tgccgtgaaa cagcttcagg
agttcattcc taacatcaag gacagggcca ccagcacaat caagcggatg
taccgggacg acttggaacg gtttaaggaa tttaaagcac aaggtgtcgc
tattaaattt ggcaagtttt ctgtaaagga aaataagcag ttagagaaaa
atgtggaaga ctttctagcc ctgacaggca ttgagagtgc agacaagctg
ctgtacacgg acagatatcc tgaggaaaaa tctgtgatca ccaacttaaa
aaggagatac tcgtttagat tacacattgg taggaacatt gcccggccct
ggaaacttat atactatcga gcaaagaaga tgttcgatgt caacaattac
aaaggcaggt atagcgaagg agatactgag aagttaaaga tgtaccattc
tctccttggg aatgactgga agacgattgg tgagatggtg gcccgaagta
gcctctccgt ggccctcaag ttctcacaga tcagcagtca aagaaatcgt
ggtgcttgga gtaagtctga aacccggaaa ctaatcaagg ctgtcgaaga
agtgattctg aagaagatgt ctccccagga gttaaaagag gtggattcca
aactccaaga aaatcctgaa agttgcctat caattgttcg ggaaaaactc
tacaagggca tatcttgggt agaagtagaa gctaaagtgc aaaccagaaa
ttggatgcag tgtaaaagta agtggacaga aattctaacc aagaggatga
ctaatggtcg gcgtatctac tatggcatga atgccctgcg ggccaaggtc
agccttattg aaaggttgta tgaaataaat gtggaagata ctaatgaaat
agactgggaa gatcttgcta gtgccatagg tgatgttcct ccatcttacg
ttcaaactaa attttctagg ctgaaagctg tctatgttcc attttggcag
aaaaagactt ttccagagat catcgactac ctttatgaga cgactctacc
tttgctgaag gaaaagttag aaaaaatgat ggagaaaaaa ggcactaaaa
tccagactcc tgcagcaccc aagcaagttt tcccatttcg agacatcttt
tattatgaag acgatagtga aggagaggac atagaaaaag aaagcgaagg
ccaggcgcca tgcatggctc acgcctgtaa ttccagtact ttgggaggcc
aaggccggtg gatcatctga ggtcaggagt tcgagaccgg cctgaccaac
atggtgaaga cctgtcacta ttaaaaatgc gaaaattagc cgggtgtggt
agtgcacacc tgtaatttca actacttggg aggctgaggc aggagaattg
cttgaaccca ggaggtggag gttgcagtga gccaagatcg caccaccgca
tgagagagag agattactat ttcttgtccc tttttctcag tttgattata
tttatataca tatgtcagta aatctgtttt cagtattgat gtttaataaa
gaatgtacaa tggccagagt tctactcttt cctctggagc attaaaatat
attgccattc ctattaaaac gtatttgaat gtgaaaa
Yeast Ydr026C
SEQ ID NO: 3
MDSVSNLKST NFQNNNDPKE SVEEAVLRYV GVDLKNHIKK TKKKLKKQKK
RKHGSKMSHE DEDTDMDWYL KTSGSKDLRK VDDIEPNSVA VAAVAAAYNS
SMREKDKRSC HKKSSNSRSE RKKHRKRKSS KERKAKIKMV LDPQLTTLDD
GITTTAFLPD DLIAETAFDK YVDTEKAYLA KHPSKSLEVN EDDKENNFNN
NSSTLVRIYT DLEGIPNDGS YIKRTPKIPE KDVKSDDLIL APEENNGDTA
LLRSDIVKAS VIDGAITKSI GKKFTPSEEN ALDQFIEEYM KIRGLDRRQM
CERIWSTDGV IRDGFWANIS KVLPYRTRSS IYKHIRRKYH IFEQRGKWTP
EEDQELARLC LEKEGHWTEV GKLLGRMPED CRDRWRNYMK CGSKRGSKRW
SKEEEELLTT VVNEMIEEAH QYQRMKALEA ANKNDRYNQM YSRGPKGKRI
SDNPTFKDMI NWTVVSERMS GTRSRIQCRY KWNKLVTDEA ARSMLSIPVS
ERKWLLERLK QLPKTSYSNI DWNSIATYKP GYPRTGLELR LCYEQMREKI
HDFKGRSTAE IIDSLLEQIN
Yeast YDR026C DNA
SEQ ID NO: 4
atggacagcg tgtcaaacct taagagcaca aattttcaga acaataatga
cccaaaagaa tctgtagaag aagcagtgct aaggtatgtg ggagtcgact
tgaagaatca tattaaaaag acaaagaaga aattgaagaa acagaagaag
agaaagcatg gttcaaaaat gtctcatgaa gatgaagaca ctgatatgga
ttggtatttg aaaacatcgg gttcaaaaga tttacgtaaa gtggatgata
tcgaaccaaa ttctgttgct gtagcagctg ttgctgccgc atacaacagc
tcaatgagag aaaaagataa gaggagctgt cataaaaaat cttctaatag
tagatcagag aggaagaagc accgtaaaag aaaaagctct aaagaaagaa
aagccaaaat aaaaatggtc ctagatccgc agctgactac actggacgat
ggcataacga caacagcatt tttacctgat gatctcattg ctgaaacagc
ttttgataag tatgttgata ctgaaaaggc atacctagct aaacatccaa
gtaaatcgtt agaagttaat gaggatgata aagaaaacaa ctttaataac
aatagcagca cgctggttag aatttatacc gatttagagg ggataccaaa
tgacggcagc tacataaagc gcactccaaa gataccagaa aaggatgtga
aatcagacga cttgatattg gcaccagaag aaaacaatgg agataccgca
ttactgcgat ccgatattgt gaaagcgtca gtaattgacg gtgctatcac
aaaatccatc ggtaagaagt tcactcccag cgaggaaaat gctttggacc
aattcatcga agaatacatg aaaataagag gcttggatag acgccaaatg
tgcgagagaa tttggtctac agacggagtc attagagatg ggttttgggc
aaatatcagc aaagttttac cttatagaac aaggtcctca atatataaac
atataagaag aaaatatcat attttcgaac agcgtgggaa gtggacgcca
gaggaagatc aagaattggc aagattatgt ttagaaaaag agggtcattg
gaccgaagta ggcaaattgt taggcagaat gcctgaagat tgtagggacc
gttggagaaa ctatatgaag tgtggttcaa agcgaggttc taaaagatgg
tcgaaagaag aagaagaatt gttgactact gtagtaaatg aaatgattga
agaggcacac caatatcaaa ggatgaaggc attggaggct gccaacaaaa
atgatagata caatcaaatg tattctaggg gtccaaaggg aaagagaatt
agcgataatc ctaccttcaa agatatgatc aattggactg tcgtcagtga
acgcatgagc ggaactagat cccgtataca gtgccgctat aagtggaata
aattagtaac agatgaagca gccagaagta tgttgagcat tcctgtttct
gaaaggaagt ggctgctaga acgattgaaa cagctaccta aaacgtccta
ttcaaatatc gactggaaca gtatagctac ctacaagccc ggatatcctc
gaactggtct tgaactgaga ttatgttacg agcagatgag agaaaagatt
catgacttta aaggaaggtc aacagctgaa ataattgatt cattgttgga
acaaatcaat taa
TTF1 (variant)
SEQ ID NO: 5
MEGESSRFEI HTPVSDKKKK KCSIHKERPQ KHSHEIFRDS SLVNEQSQIT
RRKKRKKDFQ HLISSPLKKS RICDETANAT STLKKRKKRR YSALEVDEEA
GVTVVLVDKE NINNTPKHFR KDVDVVCVDM SIEQKLPRKP KTDKFQVLAK
SHAHKSEALH SKVREKKNKK HQRKAASWES QRARDTLPQS ESHQEESWLS
VGPGGEITEL PASAHKNKSK KKKKKSSNRE YETLAMPEGS QAGREAGTDM
QESQPTVGLD DETPQLLGPT HKKKSKKKKK KKSNHQEFEA LAMPEGSQVG
SEVGADMQES RPAVGLHGET AGIPAPAYKN KSKKKKKKSN HQEFEAVAMP
ESLESAYPEG SQVGSEVGTV EGSTALKGFK ESNSTKKKSK KRKLTSVKRA
RVSGDDFSVP SKNSESTLFD SVEGDGAMME EGVKSRPRQK KTQACLASKH
VQEAPRLEPA NEEHNVETAE DSEIRYLSAD SGDADDSDAD LGSAVKQLQE
FIPNIKDRAT STIKRMYRDD LERFKEFKAQ GVAIKFGKFS VKENKQLEKN
VEDFLALTGI ESADKLLYTD RYPEEKSVIT NLKRRYSFRL HIGRNIARPW
KLIYYRAKKM FDVNNYKGRY SEGDTEKLKM YHSLLGNDWK TIGEMVARSS
LSVALKFSQI SSQRNRGAWS KSETRKLIKA VEEVILKKMS PQELKEVDSK
LQENPESCLS IVREKLYKGI SWVEVEAKVQ TRNWMQCKSK WTEILTKRMT
NGRRIYYGMN ALRAKVSLIE RLYEINVEDT NEIDWEDLAS AIGDVPPSYV
QTKFSRLKAV YVPFWQKKTF PEIIDYLYET TLPLLKEKLE KMMEKKGTKI
QTPAAPKQVF PFRDIFYYED DSEGHRKRKR R
TTF coding sequence Variant 1
SEQ ID NO: 6
      1 ATGGAAGGAGAATCAAGCAGATTTGAAATCCACACTCCAGTTTCTGACAA
    51 GAAAAAGAAAAAGTGTTCTATACATAAGGAAAGACCTCAGAAACATTCCC
  101 ACGAAATTTTCAGAGACTCCTCCCTGGTGAATGAACAGTCTCAAATAACT
  151 AGGAGGAAAAAGAGGAAAAAAGATTTCCAGCATCTCATTTCTTCTCCTTT
  201 GAAAAAATCCAGAATCTGTGATGAGACTGCAAATGCCACTTCCACACTCA
  251 AAAAGAGAAAAAAGAGAAGATATAGTGCTTTGGAGGTGGACGAGGAAGCA
  300 GGTGTTACAGTTGTCCTTGTGGATAAAGAAAATATTAACAACACACCAAA
  351 GCATTTTAGAAAGGATGTTGATGTTGTTTGTGTTGATATGAGCATAGAAC
  401 AGAAGTTACCAAGAAAGCCTAAAACAGACAAATTTCAGGTACTTGCTAAG
  451 TCACATGCACATAAATCAGAAGCCCTGCACAGTAAAGTTAGGGAGAAAAA
  501 GAATAAAAAGCATCAGAGGAAAGCTGCATCCTGGGAGAGCCAGCGGGCAA
  551 GGGACACCCTGCCTCAGTCAGAATCCCACCAGGAGGAGTCCTGGCTTTCT
  601 GTGGGTCCAGGGGGTGAAATTACAGAACTACCAGCATCTGCTCATAAAAA
  651 CAAGTCTAAGAAAAAAAAGAAAAAGTCCAGTAACCGGGAATATGAGACAC
  701 TGGCCATGCCTGAAGGATCGCAAGCAGGCAGAGAGGCCGGGACTGATATG
  751 CAGGAATCCCAGCCTACTGTGGGCTTGGATGATGAAACTCCACAACTACT
  801 AGGACCTACTCACAAAAAAAAGTCTAAGAAAAAAAAGAAGAAAAAGTCCA
  851 ATCACCAGGAATTTGAGGCATTGGCCATGCCTGAAGGATCACAAGTGGGC
  901 AGTGAGGTTGGGGCTGATATGCAGGAATCCCGGCCTGCTGTGGGCCTGCA
  951 TGGTGAAACTGCAGGAATACCAGCACCTGCTTATAAAAACAAGTCTAAGA
1001 AAAAAAAGAAAAAGTCCAATCACCAGGAATTTGAGGCAGTGGCCATGCCT
1051 GAGAGCCTCGAGAGTGCATACCCTGAAGGATCACAGGTGGGCAGTGAGGT
1101 TGGGACTGTGGAAGGCAGTACAGCTCTTAAAGGGTTCAAGGAATCCAACA
1151 GTACAAAGAAGAAGTCTAAGAAAAGGAAGCTTACGTCTGTCAAAAGGGCA
1201 CGAGTGTCTGGTGATGATTTTTCAGTGCCCAGTAAGAACTCTGAGAGCAC
1251 ACTCTTTGATTCAGTAGAAGGTGATGGCGCCATGATGGAAGAAGGTGTGA
1301 AATCTAGGCCCCGACAAAAGAAAACCCAGGCCTGTTTGGCAAGCAAGCAC
1351 GTGCAAGAGGCGCCAAGGTTAGAACCTGCAAATGAAGAACACAATGTGGA
1401 AACAGCTGAAGATTCCGAAATAAGATACTTATCTGCAGATTCAGGAGATG
1451 CCGATGATTCAGATGCGGATTTGGGTTCTGCCGTGAAACAGCTTCAGGAG
1501 TTCATTCCTAACATCAAGGACAGGGCCACCAGCACAATCAAGCGGATGTA
1551 CCGGGACGACTTGGAACGGTTTAAGGAATTTAAAGCACAAGGTGTCGCTA
1601 TTAAATTTGGCAAGTTTTCTGTAAAGGAAAATAAGCAGTTAGAGAAAAAT
1651 GTGGAAGACTTTCTAGCCCTGACAGGCATTGAGAGTGCAGACAAGCTGCT
1701 GTACACGGACAGATATCCTGAGGAAAAATCTGTGATCACCAACTTAAAAA
1751 GGAGATACTCGTTTAGATTACACATTGGTAGGAACATTGCCCGGCCCTGG
1801 AAACTTATATACTATCGAGCAAAGAAGATGTTCGATGTCAACAATTACAA
1511 AGGCAGGTATAGCGAAGGAGATACTGAGAAGTTAAAGATGTACCATTCTC
1901 TCCTTGGGAATGACTGGAAGACGATTGGTGAGATGGTGGCCCGAAGTAGC
1951 CTCTCCGTGGCCCTCAAGTTCTCACAGATCAGCAGTCAAAGAAATCGTGG
2001 TGCTTGGAGTAAGTCTGAAACCCGGAAACTAATCAAGGCTGTCGAAGAAG
2051 TGATTCTGAAGAAGATGTCTCCCCAGGAGTTAAAAGAGGTGGATTCCAAA
2101 CTCCAAGAAAATCCTGAAAGTTGCCTATCAATTGTTCGGGAAAAACTCTA
2151 CAAGGGCATATCTTGGGTAGAAGTAGAAGCTAAAGTGCAAACCAGAAATT
2201 GGATGCAGTGTAAAAGTAAGTGGACAGAAATTCTAACCAAGAGGATGACT
2251 AATGGTCGGCGTATCTACTATGGCATGAATGCCCTGCGGGCCAAGGTCAG
2301 CCTTATTGAAAGGTTGTATGAAATAAATGTGGAAGATACTAATGAAATAG
2351 ACTGGGAAGATCTTGCTAGTGCCATAGGTGATGTTCCTCCATCTTACGTT
2401 CAAACTAAATTTTCTAGGCTGAAAGCTGTCTATGTTCCATTTTGGCAGAA
2451 AAAGACTTTTCCAGAGATCATCGACTACCTTTATGAGACGACTCTACCTT
2501 TGCTGAAGGAAAAGTTAGAAAAAATGATGGAGAAAAAAGGCACTAAAATC
2551 CAGACTCCTGCAGCACCCAAGCAAGTTTTCCCATTTCGAGACATCTTTTA
2601 TTATGAAGACGATAGTGAAGGAGAGGACATAGAAAAAGAAAGCGAAGGCC
2651 AGGCGCCATGCATGGCTCACGCCTGTAATTCCAGTACTTTGGGAGGCCAA
2701 GGCCGGTGGATCATCTGA
TTF coding sequence Variant 2
SEQ ID NO: 7
      1 ATGGAAGGAGAATCAAGCAGATTTGAAATCCACACTCCAGTTTCTGACAA
    51 GAAAAAGAAAAAGTGTTCTATACATAAGGAAAGACCTCAGAAACATTCCC
  101 ACGAAATTTTCAGAGACTCCTCCCTGGTGAATGAACAGTCTCAAATAACT
  151 AGGAGGAAAAAGAGGAAAAAAGATTTCCAGCATCTCATTTCTTCTCCTTT
  201 GAAAAAATCCAGAATCTGTGATGAGACTGCAAATGCCACTTCCACACTCA
  251 AAAAGAGAAAAAAGAGAAGATATAGTGCTTTGGAGGTGGACGAGGAAGCA
  301 GGTGTTACAGTTGTCCTTGTGGATAAAGAAAATATTAACAACACACCAAA
  351 GCATTTTAGAAAGGATGTTGATGTTGTTTGTGTTGATATGAGCATAGAAC
  401 AGAAGTTACCAAGAAAGCCTAAAACAGACAAATTTCAGGTACTTGCTAAG
  451 TCACATGCACATAAATCAGAAGCCCTGCACAGTAAAGTTAGGGAGAAAAA
  501 GAATAAAAAGCATCAGAGGAAAGCTGCATCCTGGGAGAGCCAGCGGGCAA
  551 GGGACACCCTGCCTCAGTCAGAATCCCACCAGGAGGAGTCCTGGCTTTCT
  601 GTGGGTCCAGGGGGTGAAATTACAGAACTACCAGCATCTGCTCATAAAAA
  651 CAAGTCTAAGAAAAAAAAGAAAAAGTCCAGTAACCGGGAATATGAGACAC
  701 TGGCCATGCCTGAAGGATCGCAAGCAGGCAGAGAGGCCGGGACTGATATG
  751 CAGGAATCCCAGCCTACTGTGGGCTTGGATGATGAAACTCCACAACTACT
  801 AGGACCTACTCACAAAAAAAAGTCTAAGAAAAAAAAGAAGAAAAAGTCCA
  851 ATCACCAGGAATTTGAGGCATTGGCCATGCCTGAAGGATCACAAGTGGGC
  901 AGTGAGGTTGGGGCTGATATGCAGGAATCCCGGCCTGCTGTGGGCCTGCA
  951 TGGTGAAACTGCAGGAATACCAGCACCTGCTTATAAAAACAAGTCTAAGA
1001 AAAAAAAGAAAAAGTCCAATCACCAGGAATTTGAGGCAGTGGCCATGCCT
1051 GAGAGCCTCGAGAGTGCATACCCTGAAGGATCACAGGTGGGCAGTGAGGT
1101 TGGGACTGTGGAAGGCAGTACAGCTCTTAAAGGGTTCAAGGAATCCAACA
1151 GTACAAAGAAGAAGTCTAAGAAAAGGAAGCTTACGTCTGTCAAAAGGGCA
1201 CGAGTGTCTGGTGATGATTTTTCAGTGCCCAGTAAGAACTCTGAGAGCAC
1251 ACTCTTTGATTCAGTAGAAGGTGATGGCGCCATGATGGAAGAAGGTGTGA
1301 AATCTAGGCCCCGACAAAAGAAAACCCAGGCCTGTTTGGCAAGCAAGCAC
1351 GTGCAAGAGGCGCCAAGGTTAGAACCTGCAAATGAAGAACACAATGTGGA
1401 AACAGCTGAAGATTCCGAAATAAGATACTTATCTGCAGATTCAGGAGATG
1451 CCGATGATTCAGATGCGGATTTGGGTTCTGCCGTGAAACAGCTTCAGGAG
1501 TTCATTCCTAACATCAAGGACAGGGCCACCAGCACAATCAAGCGGATGTA
1551 CCGGGACGACTTGGAACGGTTTAAGGAATTTAAAGCACAAGGTGTCGCTA
1601 TTAAATTTGGCAAGTTTTCTGTAAAGGAAAATAAGCAGTTAGAGAAAAAT
1651 GTGGAAGACTTTCTAGCCCTGACAGGCATTGAGAGTGCAGACAAGCTGCT
1701 GTACACGGACAGATATCCTGAGGAAAAATCTGTGATCACCAACTTAAAAA
1751 GGAGATACTCGTTTAGATTACACATTGGTAGGAACATTGCCCGGCCCTGG
1801 AAACTTATATACTATCGAGCAAAGAAGATGTTCGATGTCAACAATTACAA
1851 AGGCAGGTATAGCGAAGGAGATACTGAGAAGTTAAAGATGTACCATTCTC
1901 TCCTTGGGAATGACTGGAAGACGATTGGTGAGATGGTGGCCCGAAGTAGC
1951 CTCTCCGTGGCCCTCAAGTTCTCACAGATCAGCAGTCAAAGAAATCGTGG
2001 TGCTTGGAGTAAGTCTGAAACCCGGAAACTAATCAAGGCTGTCGAAGAAG
2051 TGATTCTGAAGAAGATGTCTCCCCAGGAGTTAAAAGAGGTGGATTCCAAA
2101 CTCCAAGAAAATCCTGAAAGTTGCCTATCAATTGTTCGGGAAAAACTCTA
2151 CAAGGGCATATCTTGGGTAGAAGTAGAAGCTAAAGTGCAAACCAGAAATT
2201 GGATGCAGTGTAAAAGTAAGTGGACAGAAATTCTAACCAAGAGGATGACT
2251 AATGGTCGGCGTATCTACTATGGCATGAATGCCCTGCGGGCCAAGGTCAG
2301 CCTTATTGAAAGGTTGTATGAAATAAATGTGGAAGATACTAATGAAATAG
2351 ACTGGGAAGATCTTGCTAGTGCCATAGGTGATGTTCCTCCATCTTACGTT
2401 CAAACTAAATTTTCTAGGCTGAAAGCTGTCTATGTTCCATTTTGGCAGAA
2451 AAAGACTTTTCCAGAGATCATCGACTACCTTTATGAGACGACTCTACCTT
2501 TGCTGAAGGAAAAGTTAGAAAAAATGATGGAGAAAAAAGGCACTAAAATC
2551 CAGACTCCTGCAGCACCCAAGCAAGTTTTCCCATTTCGAGACATCTTTTA
2601 TTATGAAGACGATAGTGAAGGACATAGAAAAAGAAAGCGAAGG
```

1-15. (canceled)

16. A method for treating a patient having a disorder related to at least one of a chronological life-span and a replicative life-span of a cell, said disorder selected from the group consisting of a metabolic disorder, an inflammatory disorder, cardiovascular disease, diabetes type 1, diabetes type 2, artherosclerosis, Alzheimer's disease, dementia, clinical depression, adipose disorders, including obesity, fat related metabolic disorders, muscular dystrophy, sarcopenia, cachexia and osteoporosis, the method comprising administering to the patient an effective amount of fujimycin.

17. A method for increasing a replicative life span of a cell, said method comprising disrupting a function of a polynucleotide or gene encoding a polypeptide comprising SEQ ID No: 1, 3 or 5, or a polypeptide having at least 70% identity thereto or a homologue thereof.

18. The method according to claim 17 wherein said cell is a stem cell.

19. The method according to claim 18, wherein said cell is a human stem cell

20. The method according to claim 18, wherein said cell is an induced pluripotent stem cell.

21. A method of increasing a chronological lifespan of a cell, said method comprising disrupting a function of a polynucleotide or gene encoding a polypeptide comprising SEQ ID No: 1, 3 or 5, or a polypeptide having at least 70% identity thereto or a homologue thereof.

22. The method according to claim 17, wherein the function of the gene or homologue is disrupted by iRNA.

23. The method according to claim 21, wherein the function of the gene or homologue is disrupted by iRNA.

24. The method according to claim 17, wherein the function of the gene is disrupted at a transcriptional/DNA level.

25. The method according to claim 21, wherein the function of the gene is disrupted at a transcriptional/DNA level.

26. A method of increasing at least one of a chronological lifespan and a replicative lifespan of a cell comprising treating said cell with fujimycin.

27. The method according to claim 17, wherein said cell is a plant or animal cell.

28. The method according to claim 21, wherein said cell is a plant or animal cell.

29. The method according to claim 24, wherein said cell is a plant or animal cell.

30. The method according to claim 17, wherein said cell is a mammalian cell.

31. The method according to claim 21, wherein said cell is a mammalian cell.

32. The method according to claim 24, wherein said cell is a mammalian cell.

33. A composition comprising fujimycin for treating an age related disorder.

34. The composition according to claim 33, wherein the disorder is related to at least one of a chronological lifespan and a replicative life-span of a cell.

35. The composition according to claim 33 wherein said disorder is selected from the group consisting of a metabolic disorder, an inflammatory disorder, cardiovascular disease, diabetes type 1, diabetes type 2, artherosclerosis, Alzheimer's disease, dementia, clinical depression, adipose disorders, including obesity, fat related metabolic disorders muscular dystrophy, sarcopenia, cachexia and osteoporosis.

36. The composition according to claim 33 further comprising a pharmaceutically acceptable diluent, excipient or carrier.


SEQUENCE LISTING (22 sequences)

SEQ ID NO: 1 - protein, 824 aa, Saccharomyces cerevisiae
MMNFFTSKSSNQDTGFSSQHQHPNGQNNGNNNSSTAGNDNGYPCKLVSSGPCASSNNGALFTNFTLQTATPTTAISQDLYAMGTTGITSENALFQMKSMNNGISSVNNNNSNTPTIITTSQEETNAGNVHGDTGGNSLQNSEDDNFSSSSTTKCLLSSTSSLSINQREAAAAAYGPDTDIPRGKLEVTIIEARDLVTRSKDSQPYVVCTFESSEFISNGPESLGAINNNNNNNNNNQHNQNQHINNNNENTNPDAASQHHNNNSGWNGSQLPSIKEHLKKKPLYTHRSSSQLDQLNSCSSVTDPSKRSSNSSSGSSNGPKNDSSHPIWHHKTTFDVLGSHSELDISVYDAAHDHMFLGQVRLYPMIHNLAHASQHQWHSLKPRVIDEVVSGDILIKWTYKQTKKRHYGPQDFEVLRLLGKGTFGQVYQVKKKDTQRIYAMKVLSKKVIVKKNEIAHTIGERNILVTTASKSSPFIVGLKFSFQTPTDLYLVTDYMSGGELFWHLQKEGRFSEDRAKFYIAELVLALEHLHDNDIVYRDLKPENILLDANGNIALCDFGLSKADLKDRTNTFCGTTEYLAPELLLDETGYTKMVDFWSLGVLIFEMCCGWSPFFAENNQKMYQKIAFGKVKFPRDVLSQEGRSFVKGLLNRNPKHRLGAIDDGRELRAHPFFADIDWEALKQKKIPPPFKPHLVSETDTSNFDPEFTTASTSYMNKHQPMMTATPLSPAMQAKFAGFTFVDESAIDEHVNNNRKFLQNSYFMEPGSFIPGNPNLPPDEDVIDDDGDEDINDGFNQEKNMNNSHSQMDFDGDQHMDDEFVSGRFEI

SEQ ID NO: 2 - DNA, 2475 bp, Saccharomyces cerevisiae
atgatgaatttttttacatcaaaatcgtcgaatcaggatactggatttagctctcaacaccaacatccaaatggacagaacaatggaaacaataatagcagcaccgctggcaacgacaacggatacccatgtaaactggtgtccagtgggccctgcgcttcatcaaataatggtgccctttttacgaattttactttacaaactgcaacgccgaccaccgctattagtcaggacttatatgcaatgggcacaacaggaataacatcagaaaatgccctttttcaaatgaagtcaatgaataatggaatatcatcagttaataataacaacagcaacacccctacgattattaccacgtcacaggaagaaactaatgctggaaatgtacatggcgataccggtggcaattctttgcaaaattctgaagatgacaacttttcttccagttctaccaccaaatgcttactctcttccacttcttcgctatcaataaatcaacgagaagcagcagcagctgcttatggtccagataccgatattcctaggggtaaactagaagttacaataatagaagcacgtgacctagtcactagatcaaaggattcacagccttatgttgtttgtacttttgagagttcagagttcatttctaatggtcctgagtcactaggcgccattaataataacaacaataacaacaacaataatcagcataatcaaaaccagcatattaacaacaacaacgaaaataccaaccctgacgctgctagccagcatcataataataacagtggttggaacggttctcagttaccatcgataaaagagcacttgaagaaaaaacccctttatacacacagatcatcttcccaattagatcagctaaactcttgctcttcagtaaccgatccgagcaaacgttcttctaattcttcgtcgggttcttcaaatggtccaaagaatgatagttcacatccaatatggcatcacaagacaacgtttgatgttttgggatctcactcggaattagatatttctgtttatgatgctgcccacgaccatatgttcttaggccaagttagactgtatccaatgattcataatttagcacatgcttcccaacaccaatggcacagtttgaaacctcgcgttattgatgaagttgtgtccggtgatattttaatcaaatggacttacaaacagacaaagaaaagacattatggcccacaagattttgaagttcttcgattattgggtaagggtacttttggccaagtctaccaagttaagaagaaagacactcaaagaatttatgcaatgaaagttctctccaagaaagttattgtcaagaaaaatgagatcgcccacacaattggcgaaagaaatatcctagtcacgacagcgtccaaatcgtccccattcattgtcggattgaagttttcctttcaaacaccaacagatctgtatttggtcactgattatatgagtggtggagaattattctggcatttacaaaaggagggccgtttttcggaagacagagcgaaattctatatcgctgagttagtcctagcgttagaacatttacacgataacgatatcgtttacagggacctaaagcctgaaaacattctactcgatgccaacggtaacatcgctctttgcgattttggtctttctaaagctgacttgaaggatagaacaaacacattttgcggcaccacggaatacctggcaccagaattgttactggacgaaaccggctacaccaaaatggtcgatttctggtctctaggtgttttgatatttgaaatgtgttgtggttggtcccctttctttgcggaaaataatcaaaaaatgtaccaaaaaattgcctttggtaaagtcaaattccccagagacgtactgtcacaagaggggaggtcttttgtaaagggtttactaaacagaaaccccaaacatagactcggtgccattgatgatggaagagaactacgagctcatccatttttcgcagatatcgactgggaggccttgaagcagaaaaaaattccaccacctttcaaacctcacctagtctcggagacggatacctcgaattttgacccagagttcacaacagcttcaacttcatacatgaacaagcaccagccgatgatgactgctaccccgctatctccagccatgcaagcaaagtttgctggtttcacctttgttgatgagtccgccatcgatgaacacgttaataacaacagaaaattcctacaaaactcgtactttatggaacctggttcctttatcccgggaaatccaaacttacctccagacgaagatgtcatcgatgatgacggggacgaggacatcaatgatggattcaaccaagagaaaaatatgaacaacagccattcgcagatggacttcgacggcgaccaacacatggatgacgaatttgtcagtggaagattcgaaatatga

SEQ ID NO: 3 - protein, 588 aa, Saccharomyces cerevisiae
MSTLSDSDTETEVVSRNLCGIVDIGSNGIRFSISSKAAHHARIMPCVFKDRVGLSLYEVQYNTHTNAKCPIPRDIIKEVCSAMKRFKLICDDFGVPETSVRVIATEATRDAINADEFVNAVYGSTGWKVEILGQEDETRVGIYGVVSSFNTVRGLYLDVAGGSTQLSWVISSHGEVKQSSKPVSLPYGAGTLLRRMRTDDNRALFYEIKEAYKDAIEKIGIPQEMIDDAKKEGGFDLWTRGGGLRGMGHLLLYQSEGYPIQTIINGYACTYEEFSSMSDYLFLKQKIPGSSKEHKIFKVSDRRALQLPAVGLFMSAVFEAIPQIKAVHFSEGGVREGSLYSLLPKEIRAQDPLLIASRPYAPLLTEKYLYLLRTSIPQEDIPEIVNERIAPALCNLAFVHASYPKELQPTAALHVATRGIIAGCHGLSHRARALIGIALCSRWGGNIPESEEKYSQELEQVVLREGDKAEALRIVWWTKYIGTIMYVICGVHPGGNIRDNVFDFHVSKRSEVETSLKELIIDDANTTKVKEESTRKNRGYEVVVRISKDDLKTSASVRSRIITLQKKVRKLSRGSVERVKIGVQFYEE

SEQ ID NO: 4 - DNA, 1767 bp, Saccharomyces cerevisiae
atgtcaacacttagcgatagtgataccgagactgaggtcgtgtcgagaaacttgtgtggaatcgtcgacataggttctaatggtattcgttttagtatatcttccaaggctgcacatcatgcaagaattatgccttgtgtttttaaagatagggttggtctttctctatacgaagttcaatataatacacatacgaacgcaaaatgccctattcccagagatattataaaagaggtttgttctgccatgaagagattcaaattaatttgcgatgattttggtgtacctgaaactagtgtcagagtaattgcaacagaagccacgcgagatgctattaacgcggatgaatttgttaatgctgtttacggtagcactggctggaaagtagaaatattaggccaggaagatgaaactagggtcggcatatatggtgttgtttcctcatttaatacagtaagaggtctatatctagatgtggcaggtggtagtactcagttatcatgggtaataagctcgcacggagaagtcaagcaatccagcaaacctgtatctttgccatatggagctggaactcttttgagaagaatgagaacagatgataatagggcacttttttatgagattaaagaagcgtacaaagatgcgattgaaaaaattggtatacctcaagaaatgattgatgacgccaagaaagaaggtggatttgacctttggacccgtgggggtggtttaagaggtatgggacatctgcttctttaccagtcggaaggttatcccatccaaacaataattaacggatatgcttgcacttatgaagaattctcgtctatgtcagattatctattcctaaaacaaaaaataccaggttcttcaaaagagcataaaatatttaaggtttctgatagaagggctttacaacttcctgccgttggtttgttcatgagtgctgtttttgaagcgattccccagatcaaagctgtacattttagtgagggtggtgttcgagagggttcactttattctcttcttccaaaagaaattcgtgcacaagatccattgctaattgcgtcccgtccttatgctccattacttactgaaaaatatctatatctattgagaacatcaatcccacaagaagatataccagaaatagtaaacgaaaggattgctcctgctttatgtaacttagcatttgttcatgcctcttatccaaaggagttacaaccaacagctgcattacatgttgctacaagagggataatagccggctgtcatggattatctcacagagctagagcgctgataggaattgctctatgtagtagatggggcggcaacattccggaatctgaagaaaaatactcccaagaattagaacaagtagttctacgcgaaggtgataaagctgaagcattgagaattgtatggtggacgaagtatattggtacgattatgtatgtgatttgcggtgttcatccaggtggtaatatcagagataacgtatttgatttccatgtttctaagcgtagtgaggtggagaccagtttaaaagaattaatcattgatgatgcaaacactacaaaggtaaaagaagaatccacgcgtaaaaatcgcgggtatgaagtggttgtgagaattagtaaggacgatcttaaaacaagtgcttccgttcgttccagaattatcacgctacaaaagaaagtacgcaagctatctagaggaagtgtagagagggttaaaattggcgtgcaattttatgaagaataa

SEQ ID NO: 5 - protein, 439 aa, Saccharomyces cerevisiae
MVTKHQIEEDHLDGATTDPEVKRVKLENNVEEIQPEQAETNKQEGTDKENKGKFEKETERIGGSEVVTDVEKGIVKFEFDGVEYTFKERPSVVEENEGKIEFRVVNNDNTKENMMVLTGLKNIFQKQLPKMPKEYIARLVYDRSHLSMAVIRKPLTVVGGITYRPFDKREFAEIVFCAISSTEQVRGYGAHLMNHLKDYVRNTSNIKYFLTYADNYAIGYFKKQGFTKEITLDKSIWMGYIKDYEGGTLMQCSMLPRIRYLDAGKILLLQEAALRRKIRTISKSHIVRPGLEQFKDLNNIKPIDPMTIPGLKEAGWTPEMDALAQRPKRGPHDAAIQNILTELQNHAAAWPFLQPVNKEEVPDYYDFIKEPMDLSTMEIKLESNKYQKMEDFIYDARLVFNNCRMYNGENTSYYKYANRLEKFFNNKVKEIPEYSHLID

SEQ ID NO: 6 - DNA, 2128 bp, Saccharomyces cerevisiae
tcttaaacacttatgggcagcaaaaaatgcgtctttcttccctcgtctgttgttttatgtagggcgtaatgatgtttgcttgtcaacaaatgaatacgtacagaagagaattctagccaaggcaattattgcatactgcaagtactgagtacgttaacgttgctagaataacattaaatgagatgtagcaatgcagatccttcctcagtaggcttaatgctccactagaatttttgaccagccactatttgcttttttcgcaatccttttcaatactcgagagcaaagacaaaaaaaataagacatgtagtgcgctgtatggaaaagaattaattagaactttacaaacgcgtgttaaacaggcatatttaagtgtttggacctaaacaatatatcgactattgaaattcttacgcaagattttttatagttggatattcatatattcttacaactctctctactttcagttttttgaagctatatgtatcattatatacgtttatggatttttcaaacctaaacaattatactgcgtaaatgtttgattaagcaataaataaaaacaaaggattggtaagggaagaccgtgagccgcccaaaagtcttcagttaactcaggttcgtattctacattagatggtcacaaaacatcagattgaagaggatcacttggatggagctacgacggatcccgaagttaaacgggtaaaattagaaaacaacgttgaagaaatacaacctgagcaggctgagaccaataaacaagagggcaccgataaagagaataaaggaaagttcgagaaagaaactgagagaataggaggatctgaagtggttacagatgtggaaaaaggaattgtcaaatttgaatttgatggtgttgaatacacattcaaagagagacccagtgtcgtagaggaaaatgaaggtaaaattgagtttagggtggtgaataatgataatactaaagaaaacatgatggtcctaactggattaaaaaacatttttcaaaagcaattaccaaaaatgcccaaagaatacattgccaggttagtctatgatcgaagtcatctttccatggctgtcattaggaagccattgactgtcgtaggtggcataacatatcgacctttcgataagagagaattcgcagaaattgttttctgtgccatcagttcgacggaacaggtacgcggttatggtgcgcatctaatgaatcacttaaaagactatgttagaaatacctcgaacataaaatattttttgacatatgcagataattacgctattggatactttaaaaagcaaggcttcactaaagaaatcacgttggataaaagtatatggatgggatatattaaagattatgaaggtggtacgctgatgcaatgttctatgttaccaagaatacgatatttggacgcaggtaagattctattattacaagaagcggccctgcgaagaaaaataagaacgatttcgaaatcgcatattgtaaggcctggtttagagcaattcaaagacttaaacaatatcaaaccgattgatccaatgactattcctggcttgaaagaagccggctggactcccgagatggatgcgttggcacaacgtcccaagcgtggtccacacgatgcagcaatacagaatatactcacagagctacaaaatcatgcagcagcttggcccttcttacaacccgttaataaagaggaggtccccgactattatgattttatcaaagagccaatggacttgagcaccatggaaataaaattagagagcaacaaatatcagaagatggaagacttcatatatgatgccagattggtgtttaacaattgccgaatgtacaatggcgagaatacgtcgtattacaagtatgctaataggctagagaaattcttcaataataaagtaaaagaaatacctgaatattctcaccttattgattaatgcgtagaagaagcttttccgctactattcctttcgaagaagaaataaatgtttagtacggcgagacgatgtgatcaattgaggttattttactacttttcctttcatttttgtaaggttttctttctttgttagtgtgacgttggtatttacctttatgtaactatat

SEQ ID NO: 7 - protein, 2470 aa, Saccharomyces cerevisiae
MEPHEEQIWKSKLLKAANNDMDMDRNVPLAPNLNVNMNMKMNASRNGDEFGLTSSRFDGVVIGSNGDVNFKPILEKIFRELTSDYKEERKLASISLFDLLVSLEHELSIEEFQAVSNDINNKILELVHTKKTSTRVGAVLSIDTLISFYAYTERLPNETSRLAGYLRGLIPSNDVEVMRLAAKTLGKLAVPGGTYTSDFVEFEIKSCLEWLTASTEKNSFSSSKPDHAKHRALLIITALAENCPYLLYQYLNSILDNIWRALRDPHLVIRIDASITLAKCLSTLRNRDPQLTSQWVQRLATSCEYGFQVNTLECIHASLLVYKEILFLKDPFLNQVFDQMCLNCIAYENHKAKMIREKIYQIVPLLASFNPQLFAGKYLHQIMDNYLEILTNAPAKKIPHLKDDKPQILISIGDIAYEVGPDIAPYVKQILDYIEHDLQTKFKFRKKFENEIFYCIGRLAVPLGPVLGKLLNRNILDLMFKCPLSDYMQETFQILTERIPSLGPKINDELLNLVCSTLSGTPFIQPGSPMEIPSFSRERAREWRNKSILQKTGESNDDNNDIKIIIQAFRMLKNIKSRFSLVEFVRIVALSYIEHTDPRVRKLAALTSCEIYVKDNICKQTSLHSLNTVSEVLSKLLAITIADPLQDIRLEVLKNLNPCFDPQLAQPDNLRLLFIALHDESFNIQSVAMELVGRLSSVNPAYVIPSIRKILLELLTKLKFSTSSREKEETASLLCTLIRSSKDVAKPYIEPLLNVLLPKFQDTSSTVASTALRTIGELSVVGGEDMKIYLKDLFPLIIKTFQDQSNSFKREAALKALGQLAASSGYVIDPLLDYPELLGILVNILKTENSQNIRRQTVTLIGILGAIDPYRQKEREVTSTTDISTEQNAPPIDIALLMQGMSPSNDEYYTTVVIHCLLKILKDPSLSSYHTAVIQAIMHIFQTLGLKCVSFLDQIIPTILDVMRTCSQSLLEFYFQQLCSLIIIVRQHIRPHVDSIFQAIKDFSSVAKLQITLVSVIEAISKALEGEFKRLVPLTLTLFLVILENDKSSDKVLSRRVLRLLESFGPNLEGYSHLITPKIVQMAEFTSGNLQRSAIITIGKLAKDVDLFEMSSRIVHSLLRVLSSTTSDELSKVIMNTLSLLLIQMGTSFAIFIPVINEVLMKKHIQHTIYDDLTNRILNNDVLPTKILEANTTDYKPAEQMEAADAGVAKLPINQSVLKSAWNSSQQRTKEDWQEWSKRLSIQLLKESPSHALRACSNLASMYYPLAKELFNTAFACVWTELYSQYQEDLIESLCIALSSPLNPPEIHQTLLNLVEFMEHDDKALPIPTQSLGEYAERCHAYAKALHYKEIKFIKEPENSTIESLISINNQLNQTDAAIGILKHAQQHHSLQLKETWFEKLERWEDALHAYNEREKAGDTSVSVTLGKMRSLHALAEWEQLSQLAARKWKVSKLQTKKLIAPLAAGARGGSGEWDMLDEYISVMKPKSPDKEFFDAILYLHKNDYDNASKHILNARDLLVTEISALINESYNRAYSVIVRTQIITEFEEIIKYKQLPPNSEKKLHYQNLWTKRLLGCQKNVDLWQRVLRIRSLVIKPKQDLQIWIKFANLCRKSGRMRLANKALNMLLEGGTILVYQIRSKPPPPVVYAQLKYIWATGAYKEALNHLIGFTSRLAHDLGLDPNNMIAQSVKLSSASTAPYVEEYTKLLARCFLKQGEWRIATQPNWRNTNPDAILGSYLLATHFDKNWYKAWHNWALANFEVISMVQEETKLNGGKNDDDDDTAVNNDNVRIDGSILGSGSLTINGNRYPLELIQRHVVPAIKGFFHSISLLETSCLQDTLRLSTLLFNFGGIKEVSQAMYEGFNLMKIENWLEVLPQLISRIHQPDPTVSNSLLSLLSDLGKAHPQALVYPLTVAIKSESVSRQKAALSIIEKIRIHSPVLVNQAELVSHELIRVAVLWHELWYEGLEDASRQFFVEHNIEKMFSTLEPLHKHLGNEPQTLSEVSFQKSFGRDLNDAYEWLNNYKKSKDINNLNQAWDIYYNVFRKITRQIPQLQTLDLQHVSPQLLATHDLELAVPGTYFPGKPTIRIAKFEPLFSVISSKQRPRKFSIKGSDGKDYKYVLKGHEDIRQDSLVMQLFGLVNTLLKNDSECFKRHLDIQQYPAIPLSPKSGLLGWVPNSDTFHVLIREHRDAKKIPLNIEHWVMLQMAPDYENLTLLQKIEVFTYALDNTKGQDLYKILWLKSRSSETWLERRTTYTRSLAVMSMTGYILGLGDRHPSNLMLDRITGKVIHIDFGDCFEAAILREKYPEKVPFRLTRMLTYAMEVSGIEGSFRITCENVMRVLRDNKESLMAILEAFALDPLIHWGFDLPPQKLTEQTGIPLPLINPSELLRKGAITVEEAANMEAEQQNETRNARAMLVLRRITDKLTGNDIKRFNELDVPEQVDKLIQQATSIERLCQHYIGWCPFW

SEQ ID NO: 8 - DNA, 7413 bp, Saccharomyces cerevisiae
atggaaccgcatgaggagcagatttggaagagtaaacttttgaaagcggctaacaacgatatggacatggatagaaatgtgccgttggcaccgaatctgaatgtgaatatgaacatgaaaatgaatgcgagcaggaacggggatgaattcggtctgacttctagtaggtttgatggagtggtgattggcagtaatggggatgtaaattttaagcccattttggagaaaattttccgcgaattaaccagtgattacaaggaggaacgaaaattggccagtatttcattatttgatctactagtatccttggaacatgaattgtcgatagaagagttccaagcagtttcaaatgacataaacaataagattttggagctggtccatacaaaaaaaacgagcactagggtaggggctgttctatccatagacactttgatttcattctacgcatatactgaaaggttgcctaacgaaacttcacgactggctggttaccttcgagggctaataccttctaatgatgtagaggtcatgagactcgctgcaaagactctgggcaagttagccgttccaggaggtacatatacctctgatttcgtggaatttgagataaagtcttgcttagaatggcttactgcctccacggaaaagaattcattctcgagttcgaagccagaccatgctaaacatgctgcgcttctgattataacagcgttggcagagaattgtccttatttactctaccaatacttgaattccatactagataacatttggagagcactaagagacccacatttggtgatcagaattgatgcgtccattacattggccaaatgtctttccaccctacgaaatagggatcctcagttaactagccagtgggtgcagagattggctacaagttgtgaatacggatttcaagtaaacacattagaatgcatccatgcaagtttgttggtttataaggaaatcttgtttttgaaggatccctttttgaatcaagtgttcgaccaaatgtgtctaaattgcatagcttatgaaaatcataaagcgaaaatgattagagaaaagatttaccagattgttcccctattagcatcgttcaatcctcaattatttgctggcaaatatttgcaccaaattatggacaactatttagagattttaaccaatgctccagcaaataaaataccacatctcaaagatgacaaaccacagattttaatatcgattggtgatattgcatatgaagtcgggcccgatatcgcaccttatgtgaaacaaattcttgattatattgaacatgatttacagacgaaattcaaattcagaaagaaatttgaaaatgaaattttctactgcatcggaagattggcagttcccttgggccccgttctaggtaaattattaaacagaaatatactggacctgatgttcaaatgccctctttccgactatatgcaggaaacgtttcaaattctgactgagagaataccatcactaggccccaaaataaatgacgagttgcttaacctagtctgttcaaccttatctggaacaccatttatccagccagggtcaccaatggagataccatcgttttcgagagaaagagcaagagaatggagaaataaaaacatcctacagaaaactggtgaaagtaacgatgataataatgatataaaaatcattatacaagcttttagaatgttaaaaaatatcaaaagcagattttcgttggtggaattcgtgagaattgttgcactttcttacattgagcatacagatcccagagtaaggaaactagctgcgttgacatcttgtgaaatttacgtcaaggataacatctgcaaacaaacatcactacactctctgaacactgtatctgaagtgttatcaaagcttctagccattacgattgcggaccctttacaagatatccgtttagaagttttaaagaatcttaatccatgtttcgatccccagttggcacaaccagataatttgagactcttgtttactgcactgcacgatgagtcgttcaatattcagtcagtagcaatggagcttgtcggtaggttgtcttccgtaaaccctgcatacgtcatcccatcgataagaaaaatactactggaactgctaacaaaattaaaattctcaacttcttctcgagaaaaggaagaaactgccagtttgttatgtactcttatcaggtcgagtaaagatgttgcgaaaccttatatcgaacctcttttaaatgttcttttaccaaaattccaagatacctcttcaacggttgcatcaactgcactgagaactataggtgagctatctgttgtagggggcgaagatatgaagatatatcttaaggatttgtttcctttaattatcaaaacatttcaggatcaatcaaactctttcaagagagaagctgcacttaaggcccttggtcaacttgcagcctcatctggttacgtgatagatcctttactcgactatcccgaattattgggtatattggtgaatatattgaagacagaaaactctcaaaatattaggagacaaacagtcactttgataggtatactgggagctatcgacccatatcgccaaaaagaacgtgaggttacctctactaccgatatatctacagaacagaacgccccgcctatcgacattgctcttctcatgcagggcatgtctccttcgaatgatgagtattataccactgttgtcattcactgcctgctaaaaatcctaaaagatccatccctatcatcttaccacactgccgtgatccaagcgattatgcatatttttcaaacccttggtctaaaatgtgtttcattcttggaccagatcatcccaactattttggacgtaatgcgtacatgctctcagtcactattagaattttacttccaacagctttgctctttgattattatcgtaaggcaacacataagacctcatgtcgattctatattccaggctatcaaagatttttcttcggttgctaagctacaaataacgcttgtaagtgttattgaagcaatatcaaaggctctggagggtgaattcaaaagattggtccctcttactctgaccttgttccttgtaattttggagaatgacaagtctagtgacaaggtcctctccagaagggtattgagactgttagaatcgtttggtcctaacttagaaggttattcgcatttgattacacccaagatagttcaaatggcagaattcaccagcgggaacctacaaaggtctgcaataattactattggcaaactggccaaggatgttgacctttttgagatgtcctcaagaattgttcactctttacttagggtactaagttcaacaacgagtgacgaactctcaaaagtcattatgaatactttaagtctactgctaatacaaatgggcacatcctttgctatcttcatccctgtcattaatgaagttttaatgaagaaacatattcaacacacaatatatgatgacttgacaaacagaatattaaacaatgatgttttacccacaaaaattcttgaagcaaatacaacggattataagcccgcggaacaaatggaggcagcagatgctggggtcgcaaaattacctataaaccaatcagttttgaaaagtgcatggaattctagccaacaaagaactaaagaagattggcaggaatggagcaaacgtctatccattcaattattaaaagagtcaccctcccatgctctaagagcttgttcaaatcttgcaagcatgtattatccactagccaaagaactttttaataccgcattcgcatgtgtttggaccgaactttatagccaatatcaagaagatttaattgggtcattatgtatagccttatcttctcccttaaatccaccagaaatacatcaaacattgttaaacctggtagaatttatggaacacgatgacaaggcattaccaataccaactcaaagcctgggcgagtatgctgaaagatgtcacgcctatgccaaagcgctacattataaagagattaaatttattaaagagcctgagaactcaactattgaatcattgatcagcattaacaaccagctgaatcaaacggatgctgcaattggtatattaaagcatgcccaacaacatcattcacttcaattaaaggagacatggtttgaaaaattagagcgttgggaagatgcactacatgcttataatgaacgtgaaaaggcaggtgatacttccgtgagcgttacactcggtaagatgagatcccttcatgcccttggcgaatgggaacagttgtcgcaattggcagctagaaagtggaaagtttcgaagctacaaactaagaagctaatagctcccttggcagctggtgctgcgtgggggttgggagagtgggatatgcttgagcaatatatcagcgttatgaaacctaaatctccagataaggaattttttgatgcaattttatacttgcacaagaatgattacgacaatgctagtaagcatatattaaacgccagagatttgcttgtgactgaaatttccgcgttgatcaatgaaagttataatagagcatatagcgttattgttagaactcaaataataacagagtttgaggaaatcatcaagtataaacaattgccacctaattccgagaaaaaacttcactatcaaaatctttggacaaaaagactgctgggctgccaaaaaaatgtcgatttatggcaaagagtgcttagagtaagatcattggtaataaagcccaagcaagacctgcaaatatggataaaatttgcaaatttgtgcagaaaatctggtagaatgaggctagcaaataaggcattgaatatgctactagaaggaggcaacgatcctagtttaccaaatacgttcaaagctcctcccccagttgtttacgcgcaactaaaatatatttgggctacaggagcttataaagaagcattaaaccacttgataggatttacatccaggttagcgcatgatcttggtttggatccgaataatatgatcgcgcaaagtgtcaaactctcaagtgcaagtactgctccgtatgttgaggaatacacaaaattattagctcgatgttttttaaagcaaggtgagtggagaatagcaacacaaccgaactggagaaacacaaatccggatgcaattcttggttcttatctattggctacacatttcgataaaaattggtacaaggcatggcataattgggccttagctaattttgaagtaatatccatggttcaggaagagactaagctcaacggaggtaagaatgatgatgatgatgacacggcagttaataatgataatgtgcggattgacggtagtatcctaggaagtggttctttgactattaatggcaacagatacccgctagagcttattcaaagacatgttgttccagcgatcaagggcttttttcattcaatatctctattagaaacaagttgtttgcaagacacgttgaggttattgactcttttatttaactttggtggtattaaagaagtctcacaagccatgtatgaaggcttcaatttgatgaaaatagagaactggcttgaagtcttaccacagttgatctctcgtatacatcagccagatcctacggtgagtaattcccttttgtcgttgctttctgatttagggaaagctcatccacaagctctcgtgtatcctttaactgtcgcgatcaagtctgaatctgtttcaagacaaaaagcggctctttcaataatagagaaaattaggattcatagtccagtcctggtaaaccaggcagaattagttagtcacgagttgatcagagtagccgttctatggcacgaattatggtatgaaggactggaagatgcgagccgccaatttttcgttgaacataacatagaaaaaatgttttctactttagaacctttacataaacacttaggcaatgagcctcaaacgttaagtgaggtatcgtttcagaaatcatttggtagagatttgaacgatgcctacgaatggttgaataactacaaaaagtcaaaagacatcaataatttgaaccaagcttgggatatttattataacgtcttcagaaaaataacacgtcaaataccacagttacaaaccttagacttacagcatgtttctccccagcttctggctactcatgatctcgaattggctgttcctgggacatatttcccaggaaaacctaccattagaatagcgaagtttgagccattattttctgtgatctcttcgaagcaaaggccaagaaaattctccatcaagggtagcgacggtaaagattataaatacgttttaaagggacatgaagatataagacaagatagccttgttatgcaattatttggtctagttaacactttgttgaagaatgattcagagtgtttcaagagacatttggatatccaacaatacccggctattccattgtcgcctaaatctggtttactaggatgggtaccaaatagtgacacattccacgttttgatcagagaacaccgtgatgccaaaaaaattccgttgaacattgaacattgggttatgttacaaatggcccccgattatgagaatttgactcttttacaaaaaattgaagtattcacgtacgctttagataatacaaaaggccaagacctttataaaatattatggttaaagagtaggtcgtcagagacatggctagaacgtagaacaacttatacgagatctttagcagttatgtccatgactggttatattctgggactaggtgatcgccatccaagcaacctgatgctagatagaatcaccggtaaagttatccacattgatttcggcgattgttttgaagctgccatcttaagagaaaagtatccagaaaaagtgccatttagactaactaggatgttaacatacgcaatggaagttagtggaattgaaggcagtttccgaattacttgtgaaaatgtcatgagagtcttaagagataataaagaatcattaatggcgatcttggaagcttttgcgcttgatcctttgatccattggggatttgatttaccgccacaaaaacttactgagcaaactggaattcctttgccgttgattaatcctagtgaattattaaggaagggggcaattactgtcgaagaagcggcaaatatggaagcagaacaacaaaatgagaccaaaaacgccagagcaatgcttgttttgagacgtattacagataaattaacgggcaatgatatcaagaggttcaatgaattagacgtccctgagcaggttgataaactgatccaacaagccacttctattgaaaggttatgtcaacattatattggatggtgcccattctggtga

SEQ ID NO: 9 - protein, 471 aa, Saccharomyces cerevisiae
MSICPHIQQVFQNEKSKDGVLKTCNAARYILNHSVPKEKFLNTMKCGTCHEINSGATFMCLQCGFCGCWNHSHFLSHSKQIGHIFGINSNNGLLFCFKCEDYIGNIDLINDAILAKYWDDVCTKTMVPSMERRDGLSGLINMGSTCFMSSILQCLIHNPYFIRHSMSQIHSNNCKVRSPDKCFSCALDKIVHELYGALNTKQASSSSTSTNRQTGFIYLLTCAWKINQNLAGYSQQDAHEFWQFIINQIHQSYVLDLPNAKEVSRANNKQCECIVHTVFEGSLESSIVCPGCQNNSKTTIDPFLDLSLDIKDKKKLYECLDSFHKKEQLKDFNYHCGECNSTQDAIKQLGIHKLPSVLVLQLKRFEHLLNGSNRKLDDFIEFPTYLNMKNYCSTKEKDKHSENGKVPDIIYELIGIVSHKGTVNEGHYIAFCKISGGQWFKFNDSMVSSISQEEVLKEQAYLLFYTIRQVN

SEQ ID NO: 10 - DNA, 1416 bp, Saccharomyces cerevisiae
atgagcatttgtccacatatacagcaagtatttcagaatgaaaagtctaaagatggggttctaaaaacgtgcaatgctgccaggtatatattaaatcattccgtacccaaggaaaaattcttaaacaccatgaaatgtggtacatgccacgaaataaactctggtgcaactttcatgtgtctacaatgtggattttgtggatgttggaaccattcgcattttctctctcacagtaaacagattggtcacatatttggtatcaactcaaataatggccttttattttgcttcaaatgtgaggactatatagggaatatcgatctgattaacgatgctatcctagcgaagtattgggacgacgtgtgcacaaagaccatggttcctagcatggaaagaagagatgggctttctggcctgatcaacatgggatccacttgtttcatgagtagtattctccaatgtctaatccataacccttactttattaggcactcaatgagtcaaattcattctaataattgtaaagtgcgttctccagataaatgtttttcatgtgcactcgataaaattgttcatgaactttatggagcgctgaatacaaagcaagcttcttcgtcatctacatctactaatcggcaaaccggattcatatatcttttaacttgtgcctggaaaatcaatcaaaatctagcagggtattcacaacaagatgctcatgaattttggcagtttataattaaccaaatccaccaaagctatgttcttgatttgccaaatgccaaggaagtcagcagagcaaataataagcagtgtgaatgcatagtgcatactgtgtttgagggctccttggaaagttctattgtgtgtccaggctgtcaaaataattcaaagacaaccattgatccattcttggatctttctctggatatcaaggataagaaaaaactttatgaatgtcttgacagtttccataaaaaagaacagttgaaggatttcaactatcattgtggggagtgtaacagcactcaagatgcaataaagcaactaggcatacacaaattaccatcggttttggttttgcaattgaaaagattcgaacacctacttaatggaagtaacagaaaactagacgattttattgaatttccaacttatttaaatatgaaaaattactgttcaacgaaggaaaaagataagcattctgaaaatggcaaggttccagacattatttacgaattaatcggtattgtttcccacaaggggacggttaatgagggacattatattgcattttgtaaaatttctggagggcaatggtttaaattcaatgattccatggtctcctctatatctcaagaagaggttttaaaggaacaggcatatttattattctacaccattcgtcaagtaaattga

SEQ ID NO: 11 - protein, 1332 aa, Saccharomyces cerevisiae
MTERIPIKNYQRTNAKALLKLTEKLFNKNFFDLYLTSQQLVVLEYLLSISSEEDKLKAWDYFLKGNIALNVEKSFPLTQEEEHHGAVSPAVDTRSDDVSSQTIKDNNNTNTNTSISNENHVENEIEDKGDNAIANEDNFVNNDESDNVEEDLFKLDLEDLKQQISGTRFIGNLSLKIRYVLWQCAIDYIYCDRNEFGDENDTEYTLLDVEEKEEEEIGKNEKPQNKEGISKFAEDEDYDDEDENYDEDSTDVKNVDDPPKNLDSISSSNIEIDDERRLVLNISISKETLSKLKTNNVEEIMGNWNKIYHSFEYDKETMIKRLKLEESDKMIEKGKKKRSRSDLEAATDEQDRENTNDEPDTNQKLPTPEGSTFSDTGNKRPKQSNLDLTVNLGIENLSLKHLLSSIQQKKSQLGISDYELKHLIMDVRKNRSKWTSDERIGQEELYEACEKVVLELRNYTEHSTPFLNKVSKREAPNYHQIIKKSMDLNTVLKKLKSFQYDSKQEFVDDIMLIWKNCLTYNSDPSHFLRGHAIAMQKKSLQLIRMIPNITIRNRADLEKEIEDMEKDKDYELDEEEEVAGSGRKGLNMGAHMLAKENGKVSEKDSSKTVKDEAPTNDDKLTSVIPEGEKEKDKTASSTVTVHENVNKNEIKENGKNEEQDMVEESSKTEDSSKDADAAKKDTEDGLQDKTAENKEAGENNEEEEDDDDEDEDEDMVDSQSYLLEKDDDRDDLEISVWKTVTAKVRAEICLKRTEYFKNGKLNSDSEAFLKNPQRMKRFDQLFLEYKEQKALESYRQKIEQNSIMKNGFGTVLKQEDDDQLQFHNDHSLNGNEAFEKQPNDIELDDTRFLQEYDISNAIPDIVYEGVNTKTLDKMEDASVDRMLQNGINKQSRFLANKDLGLTPKMNQNITLIQQIRHICHKISLIRMLQSPLSAQNSRSNPNAFLNNHIYNYTIIDDSLDIDPVSQLPTHDYKNNRELIWKFMHKNISKVAMANGFETAHPSAINMLTEIAGDYLSNLIKTLKLHHETNSLNRGTNVEMLQTTLLENGINRPDDLFSYVESEFGKKTKKLQDIKQKLESFLRALLRPTLQELSERNFEDESQSFFTGDFASELTGEDFFGFRELGLEKEFGVLSSSVPLQLLTTQFQTVDGETKVQAKKIQPEESDSIVYKKITKGMLDAGSFWNTLLPLLQKDYERSKAYIAKQSKSSANDKTSMTSTEDNSFALLEEDQFVSKKTATKARLPPTGKISTTYKKKPIASAFILPEEDLENDVKADPTTTVNAKVGAENDGDSSLFLRTPQPLDPLDMDDAFDDTNMGSNSSFSLSLPRLNQ

SEQ ID NO: 12 - DNA, 3999 bp, Saccharomyces cerevisiae
atgactgaaagaataccaataaagaattatcaaagaacaaatgccaaagctttacttaaattgactgaaaaactttttaacaagaacttttttgatctctatttaacctctcagcaattggtcgttcttgaatacctgctgtcgatttcaagtgaagaagacaaactgaaagcatgggactatttcttaaagggaaacatagcattaaatgtcgaaaaatcatttccattaacccaagaagaagaacatcacggagcggtctctcctgccgttgacacacgatcagatgatgtatcatcacaaacaattaaggacaataacaatactaataccaacaccagtatcagcaatgaaaatcatgttgaaaatgaaattgaagataaaggcgataacgcaatagcaaatgaagataattttgtgaataatgacgaaagtgataatgttgaagaagacttattcaaattagatctagaggacttgaagcagcaaataagcggaacaaggtttattggaaacttatccttgaaaatcagatacgtcttgtggcagtgcgccatagattatatatactgtgatcgtaatgagtttggtgatgaaaatgatacagaatacaccctattagatgttgaagagaaggaggaagaggaaattggtaaaaatgagaagccacaaaacaaagaaggtatttcgaagttcgccgaggatgaagattacgacgatgaagacgagaactatgatgaagacagtacagacgtaaaaaatgtcgatgatcctccaaaaaatctcgattctatttcctcttctaatatcgaaattgacgatgaacgacgcttggtgctaaatatctcaatatcaaaagaaacactgtcaaagttaaaaacaaataatgtagaagaaattatgggaaattggaacaaaatttaccacagttttgaatacgataaagaaactatgataaagcgattaaaacttgaagaaagcgataaaatgatagagaaaggaaagaagaaacgaagtcgaagtgatttagaagcagctaccgatgaacaagatcgcgaaaatacaaatgatgagccagatactaatcaaaaattgcccactcctgaaggttcaacattcagcgatactgggaacaagcgccccaaacaaagtaatttagatttaacagtcaatctaggcatcgaaaatttatcattaaagcaccttctatcatctatccagcaaaaaaaatcccaattaggaatatcagattacgaattaaaacatctgattatggatgtcagaaaaaatcggtcaaaatggacatcggatgaaagaattgggcaagaggaattatacgaagcctgtgaaaaggttgttttggaacttagaaactacactgagcattctacaccatttctgaataaagtgagcaaaagagaagcccccaattatcatcaaatcatcaaaaagtccatggacctgaatactgttttaaaaaaactgaaaagctttcaatatgactccaaacaagaatttgtagacgatattatgctaatatggaaaaattgtttgacctataattcagatccttcacattttttgagagggcatgctattgctatgcagaagaaatctcttcagttgattcgcatgattccaaatatcacaatccgaaacagggctgatttagaaaaggaaattgaagatatggaaaaagacaaagactacgaattagatgaggaagaggaagttgctggttctggaagaaaaggattgaatatgggagctcatatgttggccaaagagaatggcaaggtgtcagaaaaagatagctctaaaaccgtcaaggatgaagcaccaaccaatgatgacaaactaacttctgtcatccctgagggggaaaaagagaaagataaaactgcttcatctactgtaacggtacacgaaaatgtaaataagaacgaaataaaagaaaatgggaaaaatgaagagcaagatatggttgaggaaagtagtaagactgaggattcatcaaaagatgctgatgctgccaaaaaggatacggaagacggactacaagataaaactgcagaaaataaggaggctggggaaaataatgaagaggaagaggatgatgatgacgaagatgaagacgaagacatggtcgactcccaatcttatttacttgaaaaggatgacgatagagacgatttggaaatatccgtgtggaaaactgtaactgccaaagttcgtgcggaaatttgcttaaaaagaactgaatattttaaaaatggaaaattaaatagtgattcagaggcgtttttgaaaaacccacaaagaatgaaaaggttcgaccagctttttcttgaatataaagagcagaaagctttagaatcatatcgtcaaaaaatagagcaaaattccattatgaaaaatggctttggaacagtactaaaacaggaagacgatgaccaattgcagtttcataatgatcactctttaaatggaaatgaagcttttgaaaagcaacccaatgatattgagttagatgataccagattcctacaggaatatgatattagtaacgccattcctgacatagtatacgagggagtaaatactaaaacattagacaagatggaagacgcttccgtggaccgcatgcttcaaaatggtatcaacaaacaaagcagatttctggctaacaaggatttaggactaacacctaaaatgaaccaaaatatcacactgattcagcaaattaggcacatatgccataaaatatccctgatcagaatgttacagagccctttatcggctcaaaactccagaagcaatcccaacgctttccttaacaaccacatttataattacactattattgatgactcactcgatattgatccggtgtcacagcttccaacgcatgattacaaaaacaacagggagctgatatggaaattcatgcataagaacatatctaaggttgctatggccaatgggtttgaaactgcccatccatcagcaataaacatgcttactgaaatcgccggggattacctatctaatctgataaagactttgaagcttcatcatgaaactaactccttaaatagaggaacaaatgtggaaatgctgcaaacaacactgttggaaaacggtatcaacaggccagacgatctattttcctatgttgaatctgaatttggtaaaaaaactaagaaacttcaggacatcaaacagaaactagaaagctttttgagagccttattaaggccaactttgcaggagttgtccgagagaaactttgaagacgagagccaaagcttttttacaggtgactttgccagcgaattgactggtgaagacttctttggttttagagagcttggattagaaaaggagtttggagttttgagttcatctgttccattacagttactgactactcagtttcaaactgttgacggggaaaccaaagtgcaggccaaaaagatccaaccggaagaatcagacagcattgtgtataagaaaattacaaaaggtatgctggatgctggttcattctggaatactctacttcccctattacaaaaagattatgaacgttccaaggcctatatagcaaagcaaagcaagtcatctgcaaatgataaaacctcaatgacttccacagaagacaattctttcgctttactagaagaggatcagtttgtctcaaagaaaaccgcaacgaaggcaagattacctcctactggtaagataagtaccacatacaaaaagaaaccgatcgcaagcgcgtttatacttccagaagaagacttggaaaacgacgtaaaagcggatccaacaacaactgtaaacgccaaagtgggtgcagaaaatgatggagattcttccttatttttgcgaacgcctcaacctttagatcctttggatatggatgatgcttttgatgataccaatatgggcagcaatagttcatttagcttgagccttcctcgccttaatcaataa

SEQ ID NO: 13 - protein, 602 aa, Saccharomyces cerevisiae
MDEVDDILINNQVVDDEEDDEEMLSGLENDSKQDLEGNDDGGEDEEDDDDDDEDDDDDEDEREDDDEQEDDDGEDDAARMDKTATPTNEHQHDEQKAAAAGAGGAGDSGDAVTKIGSEDVKLSDVDGGVGSREASSSTHEASANGEVYEYYKHMLNAAQIADSYNIYPTAAIPIQTHVNALAVSRGLKYLFLGGSDGYIRKYDLLNTLEGKLSLTILQKHSLAESIQNAGILQSYWENEIPQKKSEMKLSANKTDYEPKVSPVHSLEVQSECLFILSGLQNGGITMQGVRYMEGSIAHYFKGRNGHTQIVNILRLNGQEDRFLSGSWDKRLLEWDLQTGDIVNEFKKSRSELSSLEMRPLYSSVDVSGNVNSGKENENADDDMDSLFGDEDEDEKQDAGNEPVETGDGSNGEENKEQISEESLNIVYDESVFMTSGLNGSVHIWDRRMTQSPALSLERGAGVPPWCLSACWGVDGDHVYAGRRNACVEQFDLKMPSKPIHNLKLPSISGPVSCVKAMPNNKHLLCASRDNIRLYNVEIAVDASNSTTKSSKVPFLIVPGHHGGIISNLYLDPTSRFIISTSGNRGWQGNSTDTTLIYDIDLE

SEQ ID NO: 14 - DNA, 1809 bp, Saccharomyces cerevisiae
atggacgaggttgacgatattctaattaacaaccaggtggtggatgacgaggaggatgacgaagagatgctgagtgggctggaaaacgactcaaagcaggacctcgaggggaatgatgacggtggtgaagatgaagaggatgacgatgatgatgatgaggacgatgatgatgacgaggacgaacgagaggacgacgatgaacaggaggacgacgatggtgaggacgacgccgcaagaatggataagactgctacaccgacgaatgagcaccagcatgatgagcaaaaggctgctgctgctggtgctggcggtgcaggcgatagtggcgatgctgttactaagattggatccgaggatgtgaaattgagcgatgttgatggaggagtggggtccagggaagcatcttcctctacacacgaagcctctgctaatggagaggtttatgagtactataagcacatgttgaatgccgcacagattgcggattcgtacaatatctaccccacggcagccatacccatccagacgcacgtcaatgcgttggccgtgtccaggggtctcaagtacctgtttttgggcggtagcgatggatacataaggaagtacgacttgctgaacacgcttgaggggaaactttctctaactatcctgcagaagcattcgttggctgagtctattcagaacgcgggtatcttgcagtcgtactgggaaaatgagatcccgcagaaaaaatcagaaatgaaactctccgctaataagacagattacgagcccaaagttagccccgttcattctttggaagtccaaagcgaatgcctctttatactgagcgggctacagaatggtgggattaccatgcagggcgttcgctacatggaggggagcattgcgcactattttaagggcaggaatggacatacccaaatcgttaacatactgagattaaacggtcaagaggacaggtttttgagtggttcctgggataagcgtcttttggaatgggatttgcagacgggtgacatagttaatgagtttaaaaaatcaaggtctgaattgtcatctttggaaatgcggccgctgtactcgtccgtggatgtgtccggtaacgtcaacagtggtaaagagaatgaaaatgcagatgacgatatggattctctgtttggtgatgaagacgaagacgaaaagcaagatgctggcaacgaacccgtcgagacgggggatggttctaatggtgaagagaacaaagaacagatatctgaagaatctttgaacatagtctatgatgaatccgtttttatgacctcagggttgaacggttccgtgcatatttgggaccgacgcatgacgcagtcgccagcattgtctctggagagaggtgcaggcgtcccaccgtggtgtttgtccgcatgttggggtgtagatggtgatcatgtgtatgcagggagaaggaatgcctgtgtggagcagtttgacttgaagatgccctcgaaacctatccataatttgaaactgccttctatttcagggcctgtctcttgtgttaaagccatgcctaataacaagcatttactatgtgcatcgcgggataatatcagattgtacaacgttgaaattgcagtagatgcttcgaattcgactacaaagagttctaaagtgccgttcctcatcgtgccgggccatcacggtggtattatatcaaacttatacctcgaccccacttcaagatttataataagcacaagtggcaacagaggctggcaggggaattctacggacacgacccttatttacgatatagacttagaatag

SEQ ID NO: 15 - protein, 633 aa, Saccharomyces cerevisiae
MSSNNNTNTAPANANSSHHHHHHHHHHHHHGHGGSNSTLNNPKSSLADGAHIGNYQIVKTLGEGSFGKVKLAYHTTTGQKVALKIINKKVLAKSDMQGRIEREISYLRLLRHPHIIKLYDVIKSKDEIIMVIEYAGNELFDYIVQRDKMSEQEARRFFQQIISAVEYCHRHKIVHRDLKPENLLLDEHLNVKIADFGLSNIMTDGNFLKTSCGSPNYAAPEVISGKLYAGPEVDVWSCGVILYVMLCRRLPFDDESIPVLFKNISNGVYTLPKFLSPGAAGLIKRMLIVNPLNRISIHEIMQDDWFKVDLPEYLLPPDLKPHPEEENENNDSKKDGSSPDNDEIDDNLVNILSSTMGYEKDEIYESLESSEDTPAFNEIRDAYMLIKENKSLIKDMKANKSVSDELDTFLSQSPPTFQQQSKSHQKSQVDHETAKQHARRMASAITQQRTYHQSPFMDQYKEEDSTVSILPTSLPQIHRANMLAQGSPAASKISPLVTKKSKTRWHFGIRSRSYPLDVMGEIYIALKNLGAEWAKPSEEDLWTIKLRWKYDIGNKTNTNEKIPDLMKMVIQLFQIETNNYLVDFKFDGWESSYGDDTTVSNISEDEMSTFSAYPFLHLTTKLIMELAVNSQSN

SEQ ID NO: 16 - DNA, 1902 bp, Saccharomyces cerevisiae
atgagcagtaacaacaacacaaacacagcacctgccaatgcaaattctagccaccaccaccaccatcaccaccatcaccaccaccatcacggtcatggcggaagcaactcgacgctaaacaatcccaagtcgtccttagcggatggtgcacatatcgggaactaccaaatcgtcaaaacgctgggagaggggtcctttggtaaagttaaattggcatatcataccactacgggccaaaaagttgctctaaaaatcattaataagaaggttttggcaaagagtgatatgcagggcagaattgaaagagaaatatcttatctgagactcttaagacacccccacatcatcaaactgtatgatgttatcaaatccaaagatgaaatcattatggttatagagtacgccgggaacgaattgtttgactatattgttcagagagacaaaatgagcgagcaagaggcaagaagatttttccagcagatcatcagtgccgtcgagtactgccataggcacaaaattgtccatagagatctgaagcctgaaaacttactactagatgagcatctgaatgtaaagattgccgattttggtttgtcaaacatcatgactgatggtaatttcttaaagacttcttgtggttctcccaattatgcggctcctgaagttatcagcggtaagctgtacgcaggcccagaagtggacgtgtggtcatgtggggttatcctttatgttatgctttgtcgtcgtctaccgtttgacgatgaaagcatcccagtgcttttcaagaatatcagcaacggtgtttacaccttgcctaaatttttatctcctggagctgctgggctaatcaaaagaatgttaatcgttaatccattgaacagaataagcattcatgaaattatgcaagacgattggttcaaagttgacctgccagaatatctacttccaccagatttgaaaccacacccagaagaagagaatgaaaataatgactcaaaaaaggatggcagcagcccagataacgatgaaattgatgacaaccttgtcaatattttatcatcgaccatgggttacgaaaaagacgagatttatgagtccttagaatcatcagaagacactcctgcattcaacgaaattagggacgcgtacatgttgattaaggagaataaatctttgatcaaggatatgaaggcaaacaaaagcgtcagtgatgaactggatacctttctgtcccagtcacctccaacttttcaacaacaaagcaaatcccatcaaaagagtcaagtagatcatgaaactgccaagcaacacgcaagaaggatggcaagtgctatcactcaacaaaggacatatcaccaatcacccttcatggatcagtataaagaagaagactctacagtttccattttgcctacatctttacctcagatccacagagctaatatgttagcacaaggttcgccagctgcctctaaaatatctcctcttgtaacgaaaaaatctaaaacgagatggcattttggtatacgatctcgctcatatccattagacgttatgggtgaaatttatattgccttgaagaatttgggtgccgaatgggccaagccatctgaagaggatttatggactatcaaattaaggtggaaatatgatattggaaacaagacaaacactaatgaaaaaatacctgatttaatgaaaatggtaattcaattatttcaaattgaaaccaataattatttggtggatttcaaatttgacggctgggaaagtagttatggagatgatactactgtttctaatatttctgaagatgaaatgagtactttttcagcctacccatttttacatttaacaacaaaactaattatggaattagccgttaacagtcaaagcaattga

SEQ ID NO: 17 - protein, 713 aa, Saccharomyces cerevisiae [sequence omitted]

SEQ ID NO: 18 - DNA, 2142 bp, Saccharomyces cerevisiae
atgtcgccctctgccgtacaatcatcaaaactagaagaacagtcaagtgaaattgacaagttgaaagcaaaaatgtcccagtctgccgccactgcgcagcagaagaaggaacatgagtatgaacatttgacttcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagactcctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggactcctgcgtacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgttacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatccggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 19 - protein, 1119 aa, Saccharomyces cerevisiae
MTERIPIKNYQRTNAKALLKLTEKLFNKNFFDLYLTSQQLVVLEYLLSISSEEDKLKAWDYFLKGNIALNVEKSFPLTQEEEHHGAVSPAVDTRSDDVSSQTIKDNNNTNTNTSISNENHVENEIEDKGDNAIANEDNFVNNDESDNVEEDLFKLDLEDLKQQISGTRFIGNLSLKIRYVLWQCAIDYIYCDRNEFGDENDTEYTLLDVEEKEEEEIGKNEKPQNKEGISKFAEDEDYDDEDENYDEDSTDVKNVDDPPKNLDSISSSNIEIDDERRLVLNISISKETLSKLKTNNVEEIMGNWNKIYHSFEYDKETMIKRLKLEESDKMIEKGKKKRSRSDLEAATDEQDRENTNDEPDTNQKLPTPEGSTFSDTGNKRPKQSNLDLTVNLGIENLSLKHLLSSIQQKKSQLGISDYELKHLIMDVRKNRSKWTSDERIGQEELYEACEKVVLELRNYTEHSTPFLNKVSKREAPNYHQIIKKSMDLNTVLKKLKSFQYDSKQEFVDDIMLIWKNCLTYNSDPSHFLRGHAIAMQKKSLQLIRMIPNITIRNRADLEKEIEDMEKDKDYELDEEEEVAGSGRKGLNMGAHMLAKENGKVSEKDSSKTVKDEAPTNDDKLTSVIPEGEKEKDKTASSTVTVHENVNKNEIKENGKNEEQDMVEESSKTEDSSKDADAAKKDTEDGLQDKTAENKEAGENNEEEEDDDDEDEDEDMVDSQSYLLEKDDDRDDLEISVWKTVTAKVRAEICLKRTEYFKNGKLNSDSEAFLKNPQRMKRFDQLFLEYKEQKALESYRQKIEQNSIMKNGFGTVLKQEDDDQLQFHNDHSLNGNEAFEKQPNDIELDDTRFLQEYDISNAIPDIVYEGVNTKTLDKMEDASVDRMLQNGINKQSRFLANKDLGLTPKMNQNITLIQQIRHICHKISLIRMLQSPLSAQNSRSNPNAFLNNHIYNYTIIDDSLDIDPVSQLPTHDYKNNRELIWKFMHKNISKVAMANGFETAHPSAINMLTEIAGDYLSNLIKTLKLHHETNSLNRGTNVEMLQTTLLENGINRPDDLFSYVESEFGKKTKKLQDIKQKLESFLRALLRPTLQELSERNFEDESQSFFTGDFASELTGEDFF

SEQ ID NO: 20 - DNA, 3357 bp, Saccharomyces cerevisiae
atgactgaaagaataccaataaagaattatcaaagaacaaatgccaaagctttacttaaattgactgaaaaactttttaacaagaacttttttgatctctatttaacctctcagcaattggtcgttcttgaatacctgctgtcgatttcaagtgaagaagacaaactgaaagcatgggactatttcttaaagggaaacatagcattaaatgtcgaaaaatcatttccattaacccaagaagaagaacatcacggagcggtctctcctgccgttgacacacgatcagatgatgtatcatcacaaacaattaaggacaataacaatactaataccaacaccagtatcagcaatgaaaatcatgttgaaaatgaaattgaagataaaggcgataacgcaatagcaaatgaagataattttgtgaataatgacgaaagtgataatgttgaagaagacttattcaaattagatctagaggacttgaagcagcaaataagcggaacaaggtttattggaaacttatccttgaaaatcagatacgtcttgtggcagtgcgccatagattatatatactgtgatcgtaatgagtttggtgatgaaaatgatacagaatacaccctattagatgttgaagagaaggaggaagaggaaattggtaaaaatgagaagccacaaaacaaagaaggtatttcgaagttcgccgaggatgaagattacgacgatgaagacgagaactatgatgaagacagtacagacgtaaaaaatgtcgatgatcctccaaaaaatctcgattctatttcctcttctaatatcgaaattgacgatgaacgacgcttggtgctaaatatctcaatatcaaaagaaacactgtcaaagttaaaaacaaataatgtagaagaaattatgggaaattggaacaaaatttaccacagttttgaatacgataaagaaactatgataaagcgattaaaacttgaagaaagcgataaaatgatagagaaaggaaagaagaaacgaagtcgaagtgatttagaagcagctaccgatgaacaagatcgcgaaaatacaaatgatgagccagatactaatcaaaaattgcccactcctgaaggttcaacattcagcgatactgggaacaagcgccccaaacaaagtaatttagatttaacagtcaatctaggcatcgaaaatttatcattaaagcaccttctatcatctatccagcaaaaaaaatcccaattaggaatatcagattacgaattaaaacatctgattatggatgtcagaaaaaatcggtcaaaatggacatcggatgaaagaattgggcaagaggaattatacgaagcctgtgaaaaggttgttttggaacttagaaactacactgagcattctacaccatttctgaataaagtgagcaaaagagaagcccccaattatcatcaaatcatcaaaaagtccatggacctgaatactgttttaaaaaaactgaaaagctttcaatatgactccaaacaagaatttgtagacgatattatgctaatatggaaaaattgtttgacctataattcagatccttcacattttttgagagggcatgctattgctatgcagaagaaatctcttcagttgattcgcatgattccaaatatcacaatccgaaacagggctgatttagaaaaggaaattgaagatatggaaaaagacaaagactacgaattagatgaggaagaggaagttgctggttctggaagaaaaggattgaatatgggagctcatatgttggccaaagagaatggcaaggtgtcagaaaaagatagctctaaaaccgtcaaggatgaagcaccaaccaatgatgacaaactaacttctgtcatccctgagggggaaaaagagaaagataaaactgcttcatctactgtaacggtacacgaaaatgtaaataagaacgaaataaaagaaaatgggaaaaatgaagagcaagatatggttgaggaaagtagtaagactgaggattcatcaaaagatgctgatgctgccaaaaaggatacggaagacggactacaagataaaactgcagaaaataaggaggctggggaaaataatgaagaggaagaggatgatgatgacgaagatgaagacgaagacatggtcgactcccaatcttatttacttgaaaaggatgacgatagagacgatttggaaatatccgtgtggaaaactgtaactgccaaagttcgtgcggaaatttgcttaaaaagaactgaatattttaaaaatggaaaattaaatagtgattcagaggcgtttttgaaaaacccacaaagaatgaaaaggttcgaccagctttttcttgaatataaagagcagaaagctttagaatcatatcgtcaaaaaatagagcaaaattccattatgaaaaatggctttggaacagtactaaaacaggaagacgatgaccaattgcagtttcataatgatcactctttaaatggaaatgaagcttttgaaaagcaacccaatgatattgagttagatgataccagattcctacaggaatatgatattagtaacgccattcctgacatagtatacgagggagtaaatactaaaacattagacaagatggaagacgcttccgtggaccgcatgcttcaaaatggtatcaacaaacaaagcagatttctggctaacaaggatttaggactaacacctaaaatgaaccaaaatatcacactgattcagcaaattaggcacatatgccataaaatatccctgatcagaatgttacagagccctttatcggctcaaaactccagaagcaatcccaacgctttccttaacaaccacatttataattacactattattgatgactcactcgatattgatccggtgtcacagcttccaacgcatgattacaaaaacaacagggagctgatatggaaattcatgcataagaacatatctaaggttgctatggccaatgggtttgaaactgcccatccatcagcaataaacatgcttactgaaatcgccggggattacctatctaatctgataaagactttgaagcttcatcatgaaactaactccttaaatagaggaacaaatgtggaaatgctgcaaacaacactgttggaaaacggtatcaacaggccagacgatctattttcctatgttgaatctgaatttggtaaaaaaactaagaaacttcaggacatcaaacagaaactagaaagctttttgagagccttattaaggccaactttgcaggagttgtccgagagaaactttgaagacgagagccaaagcttttttacaggtgactttgccagcgaattgactggtgaagacttcttt

SEQ ID NO: 21 - protein, 337 aa, Saccharomyces cerevisiae
MMDKHKYRVEIQQMMFVSGEINDPPVETTSLIEDIVRGQVIEILLQSNKTAHLRGSRSILPEDVIFLIRHDKAKVNRLRTYLSWKDLRKNAKDQDASAGVASGTGNPGAGGEDDLKKAGGGEKDEKDGGNMMKVKKSQIKLPWELQFMFNEHPLENNDDNDDMDEDEREANIVTLKRLKMADDRTRNMTKEEYVHWSDCRQASFTFRKNKRFKDWSGISQLTEGKPHDDVIDILGFLTFEIVCSLTETALKIKQREQVLQTQKDKSQQSSQDNTNFEFASSTLHRKKRLFDGPENVINPLKPRHIEEAWRVLQTIDMRHRALTNFKGGRLSSKPIIM

SEQ ID NO: 22 - DNA, 1014 bp, Saccharomyces cerevisiae
atgatggacaagcataagtatcgtgtggagattcaacagatgatgtttgtctctggtgaaattaacgacccacccgtagaaaccacatcactgatagaagatatagtgaggggtcaagtgatagaaattcttttacagtcaaacaaaacggcgcatcttaggggaagtaggagcattctccctgaagacgtcattttcttgatcagacacgacaaggccaaagtcaatcgtttgagaacatatctgtcatggaaggatttgcgtaaaaacgccaaggaccaagatgctagtgccggtgtagcgagtggcactggaaatcctggggcaggtggtgaagatgatttgaaaaaagcaggtggtggcgagaaagacgaaaaagatggtggaaacatgatgaaggtcaagaaatcccaaattaagctgccatgggaattgcagtttatgttcaatgaacatcctttagaaaataatgacgacaatgatgatatggatgaggatgaacgagaagctaatatagtcactttgaaaaggctgaaaatggctgacgatagaacacgaaacatgactaaagaggagtacgtgcattggtccgattgtcgacaggcaagttttacatttaggaagaataaaaggttcaaggactggtctggaatttcgcaattaactgaggggaaaccccatgatgatgtgattgatatactggggtttctaacttttgagattgtctgttctttgacggaaacagctctgaaaatcaaacaaagagaacaggtattacagactcaaaaggacaaatcccagcaatctagccaagataatactaactttgaatttgcatcatccacattacatagaaagaaaagattatttgatggacctgaaaatgttataaacccgctcaaaccaaggcatatagaggaagcctggagagtactacaaacaattgacatgaggcatagggctttgaccaactttaaaggtggtagactcagttctaaaccaattatcatgtaa